SEQUENCE LISTING (19 sequences)

SEQ ID NO: 1 - protein, 4 aa, Homo sapiens
  MISC_FEATURE (2)..(2): Pro or His
  MISC_FEATURE (4)..(4): Any amino acid
GXRX

SEQ ID NO: 2 - protein, 7 aa, Homo sapiens
APFPRPG

SEQ ID NO: 3 - protein, 4 aa, Homo sapiens
  MISC_FEATURE (4)..(4): Any amino acid, preferably any amino acid other than Val, preferably Pro, Sar or Leu
GPRX

SEQ ID NO: 4 - protein, 4 aa, Homo sapiens
  MISC_FEATURE (4)..(4): Any amino acid other than Pro
GHRX

SEQ ID NO: 5 - protein, 4 aa, Homo sapiens
GPRP

SEQ ID NO: 6 - protein, 4 aa, Homo sapiens
GPRV

SEQ ID NO: 7 - protein, 7 aa, Homo sapiens
GPRPFPA

SEQ ID NO: 8 - protein, 7 aa, Homo sapiens
GPRVVAA

SEQ ID NO: 9 - protein, 8 aa, Homo sapiens
GPRPVVER

SEQ ID NO: 10 - protein, 6 aa, Homo sapiens
GPRPAA

SEQ ID NO: 11 - protein, 7 aa, Homo sapiens
GPRPPEC

SEQ ID NO: 12 - protein, 7 aa, Homo sapiens
GPRPPER

SEQ ID NO: 13 - protein, 6 aa, Homo sapiens
GPSPAA

SEQ ID NO: 14 - protein, 4 aa, Homo sapiens
GHRP

SEQ ID NO: 15 - protein, 5 aa, Homo sapiens
GHRPY

SEQ ID NO: 16 - protein, 5 aa, Homo sapiens
GHRPL

SEQ ID NO: 17 - protein, 5 aa, Homo sapiens
  MISC_FEATURE (5)..(5): carbox-termonal end comprises an amide group
GHRPY

SEQ ID NO: 18 - protein, 5 aa, Homo sapiens
GPRPG

SEQ ID NO: 19 - protein, 5 aa, Homo sapiens
YPRHG

(19) United States
(12) Patent Application Publication
(10) Pub. No.: US 2018/0140739 A1
(43) Pub. Date: May 24, 2018

(54) HAEMOSTATIC DEVICE

(71) Applicant: HAEMOSTATIX LIMITED, Nottingham, Nottinghamshire (GB)

(72) Inventors: Renata ZBOZIEN, Nottingham, Nottinghamshire (GB); John Benjamin NICHOLS, Nottingham, Nottinghamshire (GB); Jonathan HUNTER, Nottingham, Nottinghamshire (GB)

(21) Appl. No.: 15/572,895

(22) PCT Filed: May 11, 2016
(86) PCT No.: PCT/GB2016/051353

§ 371 Date: Nov. 9, 2017

(30) Foreign Application Priority Data
May 11, 2015 (GB) 1508014.6

(51) Int. Cl. A61L 17/00; A61L 31/04

(52) U.S. Cl. CPC A61L 17/005; A61L 31/047; A61L 2400/04; A61L 2300/418; A61L 2300/25

(57) ABSTRACT

A haemostatic device comprises a surgical fastener and a plurality of fibrinogen binding peptides immobilised to the fastener. The surgical fastener may be a suture. The device may prevent or reduce bleeding, such as suture hole bleeding, during surgical procedures.

This invention relates to a haemostatic device, such as a haemostatic suture, kits and methods for making the device and use of the device to prevent or reduce bleeding during surgery.

Sutures are typically formed from a thin fibre or thread and are used in surgical procedures to close wounds and join tissue. They may also be used to attach materials such as wound dressings or patches, to a patient. The process of using sutures to join tissue together, and form a surgical seam is often referred to as stitching. Stitching typically involves pressing a needle with an attached suture into a patient's tissue and then pulling the thread between edges of a wound. The trailing thread can be tied into a knot to secure the thread in place.

Suture hole bleeding is a frequent complication in vascular surgery and can result from the holes created by the needle being larger than the suture thread. This may occur if a suture is used in conjunction with a patch or dressing material, and the material is not able to sufficiently occlude the hole around the suture.

Suture hole bleeding may be reduced by using tissue adhesive as an alternative to, or in combination with, sutures. However, using tissue adhesives as an alternative to sutures is not always possible because, for example, they may not provide a strong enough bond for joining tissue together. In addition, applying both a suture and a tissue adhesive during surgery may be inconvenient and awkward. Furthermore, tissue adhesives may be unsuitable for certain types of wounds. Suture hole bleeding can be treated with haemostats or sealants, but this requires additional time and cost, and many such products are manufactured from materials derived from blood, thus potentially exposing patients to contamination.

There is a desire to provide a more effective solution to prevent or reduce suture hole bleeding.

According to the invention, there is provided a haemostatic device comprising a surgical fastener and a plurality of fibrinogen binding peptides immobilised to the fastener.

As used herein, the term “surgical fastener” refers to a means or agent for n [symbol] joining tissue, which is applied by piercing or puncturing tissue. Examples of surgical fasteners include sutures, staples and pins.

In a preferred embodiment, the haemostatic device comprises a suture with fibrinogen binding peptides immobilised to the suture.

The device may thus promote clotting in holes generated during its application, such as suture holes. It may reduce or prevent suture hole bleeding.

Haemostatic devices of the invention do not rely on the action of exogenous thrombin. The fibrinogen-binding peptides can be made synthetically and so are minimally antigenic, do not carry the risk of viral transmission, can be made more cheaply than recombinant proteins expressed in mammalian cell lines, and may be stored long-term at room temperature.

Advantageously, the haemostatic device of the invention may prevent or reduce bleeding without requiring a separate tissue sealant. This is because the haemostatic device of the invention comprises immobilised fibrinogen binding peptides, prior to its application to a patient. The haemostatic device may thus be described as preformed. This is in contrast to a situation in which a surgical fastener has been applied to a patient to join tissue, and then, subsequently, a tissue sealant is applied over the joined tissue. The invention thus encompasses a haemostatic device suitable for application to a patient, but which has not yet been applied to a patient. The haemostatic device is ready-to-use, as it does not require an initial step of applying a tissue sealant to the fastener, before application to the patient.

Preferably, the haemostatic device is sterile. It may have been sterilised by application of heat, steam, ethylene oxide or by irradiation, preferably by exposure to gamma radiation. The device may be packaged, preferably in sterile packaging.

The fastener may comprise a resorbable material. Examples of resorbable materials include polyglactin, poliglecaprone, polydioxanone, animal gut and oxidised cellulose. Alternatively, the fastener may comprise a non-resorbable material. Examples of non-resorbable materials include polypropylene, polyester, nylon, silk or steel. In a particularly preferred embodiment, the fastener comprises polypropylene.

If the fastener is a suture, the suture may have a diameter of 0.01 mm to 1 r [symbol] may be braided or monofilament.

The haemostatic device may comprise a film or coating of fibrinogen binding peptides. Preferably, the fastener is coated with immobilised fibrinogen binding peptides along a majority of its length. Most preferably, the fastener is coated with immobilised fibrinogen binding peptides substantially along the entirety of its length.

In some embodiments, the plurality of fibrinogen-binding peptides are non-covalently immobilised to the fastener. For example, the fibrinogen binding peptides may be adhered to, or adsorbed on to the fastener. This may be achieved by immobilising a haemostatic agent to the fastener.

The haemostatic agent may comprise a plurality of carriers and a plurality of fibrinogen-binding peptides immobilised to each carrier.

In a preferred embodiment, the carriers are soluble carriers. For example, the carriers may be soluble in blood plasma. The carders should be suitable for administration to a bleeding wound site. The carriers may comprise a polymer for example a protein, a polysaccharide, or a synthetic biocompatible polymer, such as polyethylene glycol, or a combination of any of these. Albumin is a preferred protein carrier. The soluble carrier or haemostatic agent may have a solubility of at least 10mg per ml of solvent, for example 10-1000 mg/ml, 33-1000 mg/ml, or 33-100 mg/ml

In a preferred embodiment, the fibrinogen-binding peptides are covalently immobilised to the carriers.

The carriers may comprise reactive groups which permit attachment of the fibrinogen-binding peptides. For example, the carriers may comprise thiol moieties or amine moieties on their surface. If the carriers are proteinaceous, the thiol or amine moieties may be provided by side chains of amino acids, for example cysteine or lysine. Alternatively, reactive groups may be added to the carrier. This is particularly advantageous if the carrier is formed from protein, such as albumin. For example, the carrier may be thiolated using a reagent such as 2-iminothiolane (2-IT) which is able to react with primary amine groups on the carrier. Alternatively cystamine may be coupled to carboxyl groups on the carrier in the presence of 1-Ethyl-3-(3-dimethylaminopropyl) carbodiimide hydrochloride (EDC) and N-hydroxysuccinimide (NHS), followed by reductive cleavage of the introduced disulphide bond.

In preferred embodiments, the fibrinogen-binding peptides are covalently im [symbol] the carrier via a spacer. A preferred spacer is a non-peptide spacer, for example comprising a hydrophilic polymer such as polyethylene glycol (PEG). In a preferred embodiment, a plurality of peptide conjugates, each comprising a fibrinogen-binding peptide linked to a thiol-reactive group (for example, a maleimide group) by a PEG spacer are reacted with a thiolated carrier (for example prepared using 2-IT or cystamine as described above). Suitable non-peptide spacers are described in WO 2013/114132.

The haemostatic agent may comprise a peptide conjugate. A suitable carrier may thus comprise one or more amino acid residues, for example a single amino acid residue, such as a lysine amino acid residue. An advantage of conjugates comprising carriers that comprise one or more amino acid residues is that they can readily be made using solid-phase peptide synthesis methods. In addition, they may be readily produced without use of immunogenic agents and may be resistant to sterilising radiation.

Each fibrinogen-binding peptide of the peptide conjugate may, independently, be attached at its carboxy-terminal end (optionally via a linker), or at its amino-terminal end (optionally via a linker), to the carrier.

In one example, the peptide conjugate may have the following general formula:

FBP-(linker)-X-(linker)-FBP

where:
FBP is a fibrinogen-binding peptide;
-(linker)- is an optional linker, preferably a non-peptide linker;
X is an amino acid, preferably a multifunctional amino acid, most preferably a tri-functional amino acid residue, such as lysine, ornithine, or arginine.

The peptide conjugate may be a dendrimer. The dendrimer may comprise a branched core and a plurality of fibrinogen-binding peptides separately covalently attached to the branched core. The branched, core may comprise one or more multifunctional amino acids. Each multifunctional amino acid, or a plurality of multifunctional amino acids, may have one or more fibrinogen binding peptides covalently attached to it.

The branched core may comprise: i) from two to ten multi-functional amino acid residues, wherein each fibrinogen-binding peptide is separately covalently attached to a multi-functional amino acid residue of the branched core; ii) a plurality of multi-fun [symbol] acid residues, wherein one or more fibrinogen-binding peptides are separately covalently attached to each of at least two adjacent multi-functional amino acid residues of the branched core; iii) a plurality of multi-functional amino acid residues, wherein two or more fibrinogen-binding peptides are separately covalently attached to at least one of the multi-functional amino acid residues of the branched core; iv) a plurality of multi-functional amino acid residues, wherein two or more multi-functional amino acid residues are covalently linked through a side chain of an adjacent multi-functional amino acid residue; or v) a single multi-functional amino acid residue, and a fibrinogen-binding peptide is separately covalently attached to each functional group of the multi-functional amino acid residue.

The multi-functional amino acid residues may comprise aria or tetra-functional amino acid residues, or tri- and tetra-functional amino acid residues, or the single multi-functional amino acid residue is a tri- or tetra-functional amino acid residue.

Each fibrinogen-binding peptide may have a different point of attachment to the branched core, so the fibrinogen-binding peptides are referred to herein as being “separately covalently attached” to the branched core.

The branched core comprises any suitable amino acid sequence. The branched core may comprise up to ten multi-functional amino acid residues, for example two to ten, or two to six multi-functional amino acid residues.

The branched core may comprise a plurality of consecutive multi-functional amino acid residues. The branched core may comprise up to ten consecutive multi-functional amino acid residues.

The term “tri-functional amino acid” is used herein to refer to any organic compound with a first functional group that is an amine (—NH₂), a second functional group that is a carboxylic acid (—COOH), and a third functional group. The term “tetra-functional amino acid” is used herein to refer to any organic compound with a first functional group that is an amine (—NH₂), a second functional group that is a carboxylic acid (—COOH), a third functional group, and a fourth functional group. The third and fourth functional group may be any functional group that is capable of reaction with a carboxy-terminal end of a fibrinogen-binding peptide, or with a functional group of a linker attached to the carboxy-terminal end of a fibrinogen-binding peptide.

Multifunctional amino acids may comprise a central carbon atom (α- or 2-) b [symbol] amino group, a carboxyl group, and a side chain bearing, a further functional group (thereby providing a tri-functional amino acid), or a further two functional groups (thereby providing a tetra-functional amino acid.

The, or each, multi-functional amino acid residue may be a residue of a proteinogenic or non-proteinogenic multi-functional amino acid, or a residue of a natural or unnatural multi-functional amino acid.

Proteinogenic tri-functional amino acids possess a central carbon atom (α- or 2-) bearing an amino group, a carboxyl group, a side chain and an α-hydrogen levo conformation. Examples of suitable tri-functional proteinogenic amino acids include L-lysine, L-arginine, L-aspartic acid, L-glutamic acid, L-asparagine, L-glutamine, and L-cysteine.

Examples of suitable tri-functional non-proteinogenic amino acid residues include D-lysine, beta-Lysine, L-ornithine, D-ornithine, and D-arginine residues.

Thus, examples of suitable tri-functional amino acid residues for use in a peptide dendrimer of the invention include lysine, ornithine, arginine, aspartic acid, glutamic acid, asparagine, glutamine, and cysteine residues, such as L-lysine, D-lysine, beta-Lysine, L-ornithine, D-ornithine, L-arginine, D-arginine, L-aspartic acid, D-aspartic acid, L-glutamic acid, D-glutamic acid, L-asparagine, D-asparagine, L-glutamine, D-glutamine, L-cysteine, and D-cysteine residues.

Examples of suitable multi-functional unnatural amino acids suitable for use in a peptide dendrimer of the invention include Citrulline, 2,4-diaminoisobutyric acid, 2,2′-diaminopimelic acid, 2,3-diaminopropionic acid, and cis-4-amino-L-proline. Multi-functional unnatural amino acids are available from Sigma-Aldrich.

In some embodiments, the branched core may comprise a homopolymeric multi-functional amino acid sequence, for example a poly-lysine, poly-arginine, or poly-ornithine sequence, such as a branched core comprising from two to ten, or from two to six, consecutive lysine, arginine, or ornithine residues. In other embodiments, the branched core may comprise different multi-functional amino acid residues, for example one or more lysine residues, one or more arginine residues, and/or one or more ornithine residues.

In other embodiments, the branched core may comprise a plurality of multi-functional amino acid residues, and one or more other amino acid residues.

Where the branched core comprises a plurality of multi-functional amino aci [symbol] adjacent multi-functional amino acid residues may be linked together by amino acid side chain links, by peptide bonds, or some adjacent multi-functional amino acid residues may be linked together by side chain links and others by peptide bonds.

In further embodiments, the branched core may comprise two or more multi-functional amino acid residues, and at least one fibrinogen-binding peptide is separately attached to each of two or more of the multifunctional amino acid residues, and two or more fibrinogen-binding peptides are separately attached to at least one of the multi-functional amino acid residues of the branched core.

According to other embodiments, two fibrinogen-binding peptides are separately attached to a terminal multi-functional amino acid residue of the branched core.

Examples of structures of peptide dendrimers include peptide dendrimers in which;
the branched core comprises a first tri-functional amino acid residue to which two fibrinogen-binding peptides are attached, and a second tri-functional amino acid residue to which one fibrinogen-binding peptide is attached;
the branched core comprises a first tri-functional amino acid residue to which two fibrinogen-binding peptides are attached, and a second tri functional amino acid residue to which two fibrinogen-binding peptides are attached;
the branched core comprises a first tri-functional amino acid residue to which two fibrinogen-binding peptides are attached, a second tri-functional amino acid residue to which one fibrinogen-binding peptide is attached, and a third tri-functional amino acid residue to which one fibrinogen-binding peptide is attached; or
the branched core comprises a first tri-functional amino acid residue to which two fibrinogen-binding peptides are attached, a second tri-functional amino acid residue to which one fibrinogen-binding peptide is attached, a third tri-functional amino acid residue to which one fibrinogen-binding peptide is attached, and a fourth tri-functional amino acid residue to which one fibrinogen-binding peptide is attached.

The peptide dendrimer may comprise the following general formula (I):

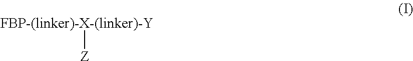

where:
FBP is a fibrinogen-binding peptide;
-(linker)- is an optional linker, preferably a non-peptide linker;
X is a tri-functional amino acid residue, preferably lysine, ornithine, or arginine;
Y is -FBP, or —NH₂;
Z is -(linker)-FBP when Y is -FBP, or -[-Xₙ-(linker)-FBP]ₐ-(linker)-FBP when y is —NH₂;

where:
Xₙ is a tri-functional amino acid residue, preferably lysine, L-ornithine, or arginine; and
a is 1-10, preferably 1-3.

For example, when Y is NH₂, Z is -[-Xₙ-(linker)-FBP]ₐ-(linker)-FBP, the structure of the dendrimer is as follows:
where a is 1:

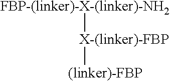

or, where a is 2:

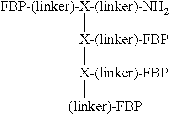

or, where a is 3:

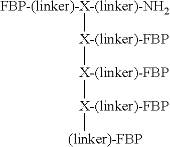

Alternatively, Z is -[-Xₙ-(linker)-FBP]ₐ-(linker)-FBP when Y is -FBP;

where;
Xₙ, is a tri-functional amino acid residue, preferably lysine, L-ornithine, or arginine; and
a is 1-10, preferably 1-3.

For example, when Y is -FBP, Z is -[-Xₙ-(linker)-FBP)]ₐ-(linker)-FBP and a is 1, the structure of the dendrimer is as follows:

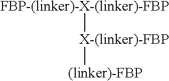

The peptide dendrimer may comprise the following general formula (II):

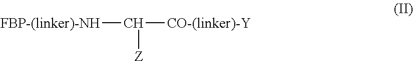

where;
FBP is a fibrinogen-binding peptide;
-(linker)- is an optional linker, preferably comprising —NH(CH₂)₅CO—;
Y is -FBP, or —NH₂;
Z is;
-R-(linker)-FBP, when Y is -FBP, or

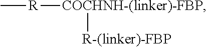

when Y is —NH₂; or

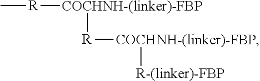

when Y is —NH₂; or

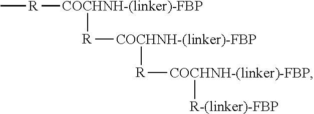

when Y is —NH₂;

where R is —(CH₂)₄NH—, —(CH₂)₃NH—, or —(CH₂)₃NHCNHNH—.

Consequently, in one embodiment, Z may be:

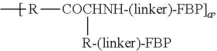

when Y is —NH₂;

where R is —(CH₂)₄NH—, —(CH₂)₃NH—, or —(CH₂)₃NHCNHNH—;

where a is 1-3.

Alternatively, a may be 4-10, or it may be 1-10.

In another embodiment, Z is:

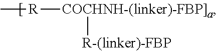

when Y is -FBP;

where R is —(CH₂)₄NH—, —(CH₂)₃NH—, or —(CH₂)₃NHCNHNH—;

where a is 1-10, preferably 1-3.

For example, Z is:

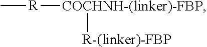

when Y is -FBP and a is 1.

The peptide dendrimer may comprise the following general formula (III):

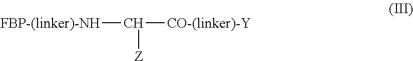

where:
FBP is a fibrinogen-binding peptide;
-(linker)- is an optional linker, preferably comprising —NH(CH₂)₅CO—;
Y is -FBP, or —NH₂;
Z is:
—(CH₂)₄NH-(linker)-FBP, when Y is -FBP; or

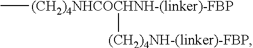

when Y is —NH₂; or

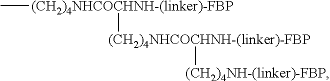

when Y is —NH₂; or

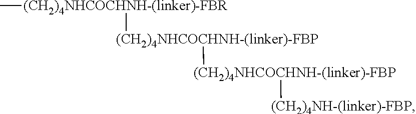

when Y is —NH₂.

Consequently, in one embodiment, Z may be:

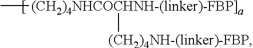

when Y is —NH₂;

where a is 1-3.

Alternatively a is 4-10, or it may be 1-10.

In another embodiment, Z is:

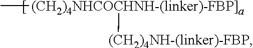

when Y is -FBP;

where a is 1-10, preferably 1-3.

For example, Z is;

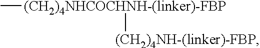

when Y is -FBP and a is 1.

One or more, or each, fibrinogen-binding peptide may be covalently attached by a non-peptide linker. The linker may be any suitable linker that does not interfere with binding of fibrinogen to fibrinogen-binding peptides. The linker may comprise a flexible, straight-chain linker, suitably a straight-chain alkyl group. Such linkers may thus allow the fibrinogen-binding peptides of the peptide dendrimer to extend away from each other. For example, the linker may comprise a —NH(CH₂)ₙCO— group, where n is any number, suitably 1-10, for example 5. A linker comprising a —NH(CH₂)₅CO— group may be formed by use of ε-amino acid 6-aminohexanoic acid (εAhx).

A particular advantage of peptide conjugates, such as peptide dendrimers, is that they can readily be sterilised, for example by exposure to irradiation, suitably gamma irradiation, without significant loss of the ability of the peptide dendrimer, or composition, to polymerise with fibrinogen.

According to the invention, there is provided a method of sterilising a haemostatic device comprising exposing the device to gamma irradiation, preferably up to 30 kGy, wherein the haemostatic device comprises a surgical fastener and a plurality of fibrinogen binding peptides immobilised to the fastener. Preferably, the fibrinogen binding peptides are provided by peptide conjugates, such as peptide dendrimers.

In theory there is no upper limit to the number of fibrinogen-binding peptides per carrier. However, in practice, for any particular structure, the number of fibrinogen-binding peptides can be varied and tested to determine the optimum number for the desired fibrinogen polymerisation properties, for example, for the speed fibrinogen polymerisation or for the density of the hydrogel produced by polymerisation with fibrinogen. The optimum number is likely to depend on many factors, such as the nature of the carrier, and the number of reactive groups on each carrier for attaching the fibrinogen-binding peptides. However, it is preferred that on average there are up to 100 fibrinogen-binding peptides per carrier molecule. Preferably, on average there are at least three, preferably at least five fibrinogen-binding peptides per carrier molecule. A preferred range is 10-20 fibrinogen-binding peptides per carrier molecule. Peptide conjugates, such as peptide dendrimers may comprise a total of up to twenty fibrinogen binding peptides per dendrimer, for example up to ten fibrinogen-binding peptides per dendrimer, or up to five fibrinogen-bin [symbol] per dendrimer.

The haemostatic agent may be immobilised to the fastener by contacting the fastener with a solution or suspension of the haemostatic agent, and drying. An example of such a process is exemplified in Examples 1 and 2.

Alternatively, the haemostatic agent may be immobilised to the fastener by heat immobilisation or thermal grafting. An example of such a process is described in Example 3.

Tseng Y., Mullins W. and Park K.; Biomaterials; 1993; 14; p392-400 describes a process for thermal grafting albumin to Polypropylene, with the aim of inhibiting the adsorption of thrombogenic proteins to the surface and decreasing platelet adhesion and activation. Albumin was grafted on to polypropylene (PP) films by thermolysis of azido groups of 4-azido-2-nitophenyl albumin (ANP-albumin). The PP film was adsorbed with ANP-albumin at the concentration of 5 mg/ml or higher and incubated at 100° C. for longer than five hours Although the albumin was denatured by the process, platelet adhesion and activation was reduced.

Surprisingly, the applicant has found that heating a solution of a haemostatic agent whilst in contact with a suture thread produces a haemostatic suture that is able to form a clot when the haemostatic suture is contacted by fibrinogen.

Consequently, the invention may thus provide a method of immobilising fibrinogen binding peptides to a substrate comprising: contacting the substrate with a solution or a suspension comprising the fibrinogen binding peptides; and heating to a temperature of 40° C. or higher, 50° C. or higher, 60° C. or higher, 70° C. or higher, 80° C. or higher, or 90° C. or higher. Preferably, the temperature is no higher than 100° C. Alternatively, the temperature is no higher than 120° C. Heating may occur up to a maximum of 24 hours. The fibrinogen binding peptides may be provided by a haemostatic agent as described in any form above. The substrate is preferably a surgical fastener. In a preferred embodiment, the substrate is manufactured from, or comprises, polypropylene.

In preferred embodiments, the plurality of fibrinogen-binding peptides are covalently immobilised to the fastener. The fibrinogen-binding, peptides may be covalently immobilised to the fastener via a spacer. The spacer may comprise a peptide spacer, comprising one or more amino acid residues. For example, the spacer may comprise one or m [symbol] residues. Alternatively, the spacer may comprise 6-aminohexanoic acid or β-alanine.

The fastener material may comprise reactive groups which permit covalent attachment of the fibrinogen-binding peptides. For example, the material may comprise thiol moieties or amine moieties on its surface. If the material is proteinaceous, the thiol or amine moieties may be provided by side chains of amino acids, for example cysteine or lysine. Alternatively, reactive groups may be added to the fastener material.

If fibrinogen binding peptides are covalently immobilised to the fastener, a preferred material is oxidised cellulose, such as oxidised regenerated cellulose. Examples of methods by which fibrinogen binding peptides can be covalently attached to oxidised cellulose are described in Example 4 below. Such methods could be used for any fastener material that has free carboxyl groups on its surface.

Alternatively, haemostatic agents described herein may be covalently immobilised to the surgical fastener. In some embodiments, carriers of the haemostatic agent may be covalently immobilised to the carrier. For example, there may be provided a haemostatic device comprising a surgical fastener with peptide conjugates or peptide dendrimers covalently immobilised to the fastener.

The term “peptide” as used herein also incorporates peptide analogues. Several peptide analogues are known to the skilled person. Any suitable analogue may be used provided fibrinogen is able to bind the fibrinogen binding peptide.

Examples of suitable fibrinogen binding peptides and how they may be identified are provided in WO 2005/035002, WO 2007/015107 and WO 2008/065388.

Preferably the fibrinogen-binding peptides are each 4-60, preferably 4-30, more preferably 4-10, amino acid residues in length. In other embodiments, each fibrinogen-binding peptide may be at least 5, 6, 7, 8, 9, 10, or 11 amino acid residues in length. It is preferred that each fibrinogen binding peptide is no longer than 60 amino acid residues in length, more preferably no longer than 30 amino acid residues in length.

Preferably each fibrinogen-binding peptide is a synthetic peptide.

Preferably each fibrinogen binding peptide binds to fibrinogen with a dissociation constant (K_{D}) of between 10⁻⁹ to 10⁻⁶ M, for example around 10, 20, 30, 40, 50, 60, 70, 80, 90, 100, 110, 120, 130, 140, 150, 200, 250, 300, 350, 400, or more nM. A K_{D} of around 100 nM is preferred. The dissociation constant can be measured at equilibrium. For ex [symbol] labelled fibrinogen of known concentration can be incubated with microspheres to which the fibrinogen binding moiety has been cross-linked. Typically 5 μM peptide is cross-linked to 1 gm microspheres, or 15-40 μmoles of peptide is cross-linked to 1 gm of microspheres. The peptide-linked microspheres are diluted to 0.5 mg/ml, and incubated in isotonic buffer at pH 7.4 (for example 0.01M Hepes buffer containing 0.15M NaCl) with radio labelled fibrinogen at concentrations of between 0.05 and 0.5mg/ml for up to 1 hr at 20° C. The fibrinogen bound to the fibrinogen binding moiety on the microspheres can be separated from the free fibrinogen by centrifugation and the amount of free and bound fibrinogen measured. The dissociation constant can then be calculated by Scatchard analysis by plotting concentration of bound fibrinogen against the ratio of the concentrations of bound: free fibrinogen, where the slope of the curve represents K_{D}.

A molecule of fibrinogen consists of three pairs of non-identical polypeptide chains, Aα, Bβ and γ, linked together by disulfide bonds. Fibrinogen chains are folded into three distinct structural regions, two distal D regions linked to one central E region. Each D region contains polymerization ‘a’ and ‘b’ holes located in the C terminus of the γ and Bβ chains, respectively. Thrombin catalyses the removal of short peptides, fibrinopeptides A (FpA) and B (FpB), from the amino-terminus of the Aα and Bβ chains of fibrinogen in the central E region, respectively, exposing two polymerisation sites: “knob A”, with amino-terminal sequence Gly-Pro-Arg-; and “knob B”, with amino-terminal sequence Gly-His-Arg-. The newly exposed polymerization knobs of one fibrin monomer interact with corresponding holes of another fibrin monomer through ‘A-a’ and ‘B-b’ knob-hole interactions, resulting in the assembly of fibrin monomers into half-staggered, double-stranded protofibrils.

In preferred embodiments of the invention, each fibrinogen binding peptide comprises the sequence Gly-(Pro,His)-Arg-Xaa (SEQ ID NO: 1) where Xaa is any amino acid and Pro/His means that either praline or histidine is present at that position. Preferably this sequence is at an amino terminal end of the peptide. For example, the peptide may comprise the sequence NH₂-Gly-(Pro,His)-Arg-Xaa (SEQ ID NO: 1). The peptide may be attached to the carrier or fastener via its carboxy-terminal end.

However, in some embodiments, the amino acid sequence may be at a carboxy-terminal end of the peptide and the peptide may be attached to the carrier or fastener via its amino-terminal end. For example, at least one fibrinogen-binding peptide that binds preferentially to hole ‘a’ over hole ‘b’ of fibrinogen, such as a peptide comprising sequence APFPRPG (SEQ ID NO: 2), may be attached via its amino-terminal end to the carrier or to the fastener. If the fibrinogen-binding peptide) is attached via its amino-terminal [symbol] carboxy-terminal end of the peptide may comprise an amide group. The presence of an amide group, rather than a carboxyl group (or a negatively charged carboxylate ion), at the exposed carboxy-terminal end of the peptide may help to optimise binding of the fibrinogen-binding peptide to fibrinogen.

In some embodiments of the invention, at least some of the fibrinogen binding peptides comprise an amino acid sequence Gly-Pro-Arg-Xaa (SEQ ID NO: 3) wherein Xaa is any amino acid. Preferably, Xaa is any amino acid other than Val, and is preferably Pro, Sar, or Leu.

In some embodiments, at least some of the fibrinogen binding peptides comprise an amino acid sequence Gly-His-Arg-Xaa (SEQ ID NO; 4), wherein Xaa is any amino acid other than Pro.

According to some embodiments of the invention, the fibrinogen-binding peptides bind preferentially to hole ‘a’ of fibrinogen over hole ‘b’ of fibrinogen. Examples of sequences of suitable fibrinogen-binding peptides that bind preferentially to hole ‘a’ over hole ‘b’ of fibrinogen include: GPR-; GPRP-(SEQ ID NO: 5); GPRV-(SEQ ID NO; 6); GPRPFPA-(SEQ ID NO; 7); GPRVVAA-(SEQ ID NO; 8); GPRPVVER-(SEQ ID NO; 9); GPRPAA-(SEQ ID NO: 10) ; GPRPPEC-(SEQ ID NO: 11); GPRPPER-(SEQ ID NO: 12); GPSPAA-(SEQ ID NO: 13).

According to some embodiments, the fibrinogen-binding peptides bind preferentially to hole ‘b’ of fibrinogen over hole ‘a’ of fibrinogen. Examples of sequences of fibrinogen-binding peptides that bind preferentially to hole ‘b’ over hole ‘a’ of fibrinogen include: GHR-, GHRP-(SEQ ID NO: 14), GHRPY-(SEQ ID NO: 15), GHRPL-(SEQ NO: 16), GHRPYamide-(SEQ ID NO: 17).

A fastener or a carrier may comprise fibrinogen-binding peptides of different sequence. For example, in some embodiments the fastener or carrier may comprise fibrinogen-binding peptides that have different selectivity of binding to hole ‘a’ over hole ‘b’ of fibrinogen.

If the haemostatic agent comprises a plurality of carriers immobilised to the fastener, the plurality of carriers may comprise a first plurality of carriers, and a second plurality of carriers, wherein the fibrinogen-binding peptides attached to the first plurality of carriers are of different sequence to the fibrinogen-binding peptides attached to the second plurality of carriers.

A haemostatic agent suitable for immobilisation to the fastener may compris [symbol] dendrimer, and a peptide conjugate comprising two or more fibrinogen-binding peptides. The peptide conjugate may comprise fibrinogen-binding peptides of the same sequence, or of different sequence. For example, the peptide conjugate may comprise only fibrinogen-binding peptides that bind preferentially to hole ‘a’ over hole ‘b’ of fibrinogen, or only fibrinogen-binding peptides that bind preferentially to hole ‘b’ over hole ‘a’ of fibrinogen, or one or more fibrinogen-binding peptides that bind preferentially to hole ‘a’ over hole ‘b’ of fibrinogen and one or more fibrinogen-binding peptides that bind preferentially to hole ‘b’ over hole ‘a’ of fibrinogen. In some embodiments, the peptide conjugate may be a peptide dendrimer. The fibrinogen-binding peptides of the peptide dendrimer may bind preferentially to hole ‘a’ of fibrinogen over hole ‘b’ of fibrinogen, and the fibrinogen-binding peptides of the peptide conjugate may bind preferentially to hole ‘b’ of fibrinogen over hole ‘a’ of fibrinogen. Such compositions have been found to have synergistic effects in that they are able to polymerise fibrinogen more rapidly than either the peptide dendrimer or the peptide conjugate alone. The mechanism of this synergistic effect is not fully understood, but without being bound by theory, it is believed that it may occur because the composition provides more ‘A’ and ‘B’ fibrinogen polymerisation sites.

Alternatively, the fibrinogen-binding peptides of the peptide dendrimer may bind preferentially to hole ‘b’ of fibrinogen over hole ‘a’ of fibrinogen, and the fibrinogen-binding peptides of the peptide conjugate bind preferentially to hole ‘a’ of fibrinogen over hole ‘b’ of fibrinogen.

Preferably, the fibrinogen binding peptides are not fibrinogen or do not comprise fibrinogen. For example, the haemostatic device may not comprise immobilised fibrinogen. The device is preferably not formed by immobilising fibrinogen (either covalently or non-covalently) or by immobilising haemostatic agents comprising fibrinogen to the fastener.

In a preferred arrangement, a fibrinogen molecule can bind at least two fibrinogen binding peptides. Consequently, if the haemostatic device comprises a plurality of immobilised carriers, with a plurality of fibrinogen binding peptides immobilised to each carrier, the fibrinogen molecules may become non-covalently cross-linked via the carriers, to form a copolymer comprising the carriers and fibrinogen which has characteristics of a fibrin clot. So, the fibrinogen binding peptides may comprise one or more sequences that can bind to two distinct regions of fibrinogen, simultaneously. For example, fibrinogen comprises two terminal domains (D-domains), each of which may bind to a fibrinogen-binding peptide.

The invention may provide a kit for formation of a haemostatic device comp [symbol] fastener and, separately, a plurality of fibrinogen binding peptides.

In a particularly preferred embodiment, the fibrinogen binding peptides of the kit are provided by a haemostatic agent, as described in any form herein.

The kit may further comprise instructions to apply the fibrinogen binding peptides to the fastener to form the haemostatic device, before application of the device to a patient.

The invention may provide a method comprising applying a haemostatic device according to the invention, to a patient. The haemostatic device may thus be used to stitch or seal a wound, to join tissue, or to attach a wound dressing to a patient. Preferably, the haemostatic device may be applied during vascular surgery.

The invention may provide a method of reducing or preventing suture hole bleeding by applying the haemostatic device, in the form of a haemostatic suture, to a patient.

The invention may provide a method of making a haemostatic device comprising immobilising a plurality of fibrinogen binding peptides to a surgical fastener.

The method may comprise non-covalently immobilising the fibrinogen binding peptides to the fastener. For example, the method may comprise immobilising a haemostatic agent to the fastener, wherein the agent comprises a plurality of carriers and wherein there are a plurality of fibrinogen-binding peptides immobilised to each carrier.

The fibrinogen binding peptide may be non-covalently immobilised to the fastener by contacting a solution or suspension comprising the fibrinogen binding peptides with the fastener, and drying. Alternatively, the method may comprise covalently immobilising fibrinogen binding peptides to the fastener.

The invention may provide a haemostatic device obtainable by a method of the invention.

Embodiments of the invention are now described by way of example only, with reference to the accompanying drawings in which:

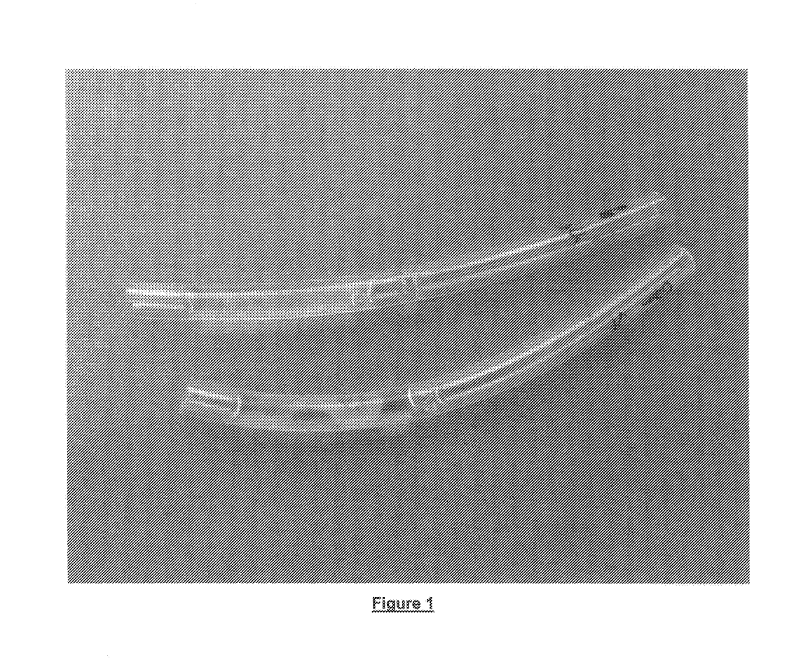

FIG. 1 shows the ability of a haemostatic suture to form a clot in human fibrinogen;

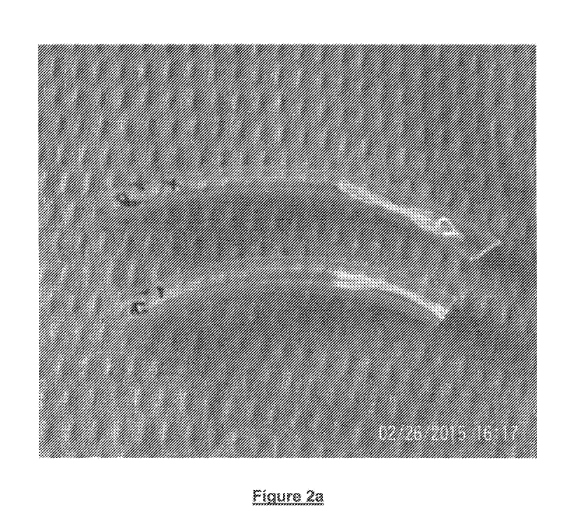

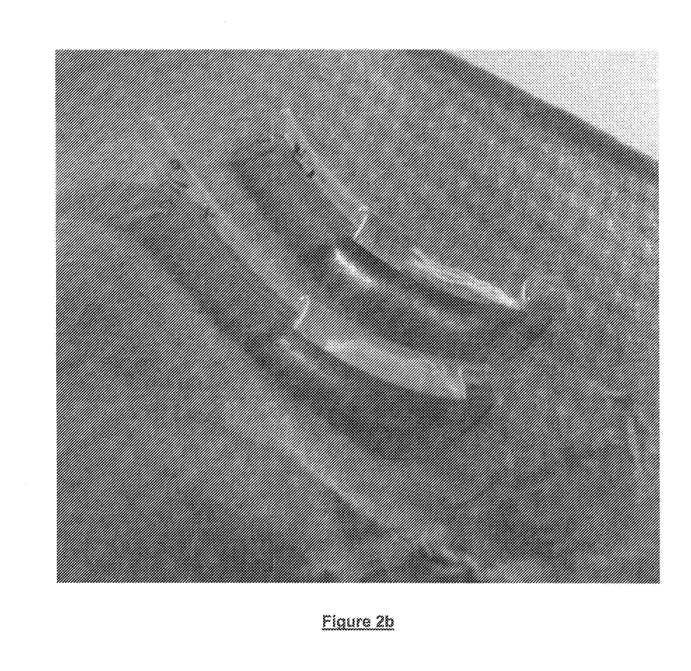

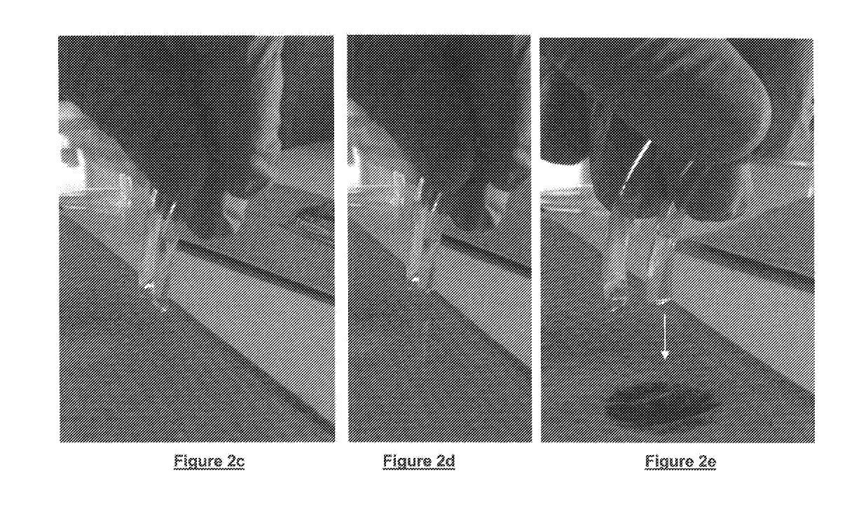

FIG. 2a shows a haemostatic suture and a control suture placed intopolypropylene tubes;

FIG. 2b shows polymerisation of fibrinogen with a haemostatic suture;

FIGS. 2c-2e show the ability of a haemostatic suture to clot fibrinogen an occlude a polypropylene tube.

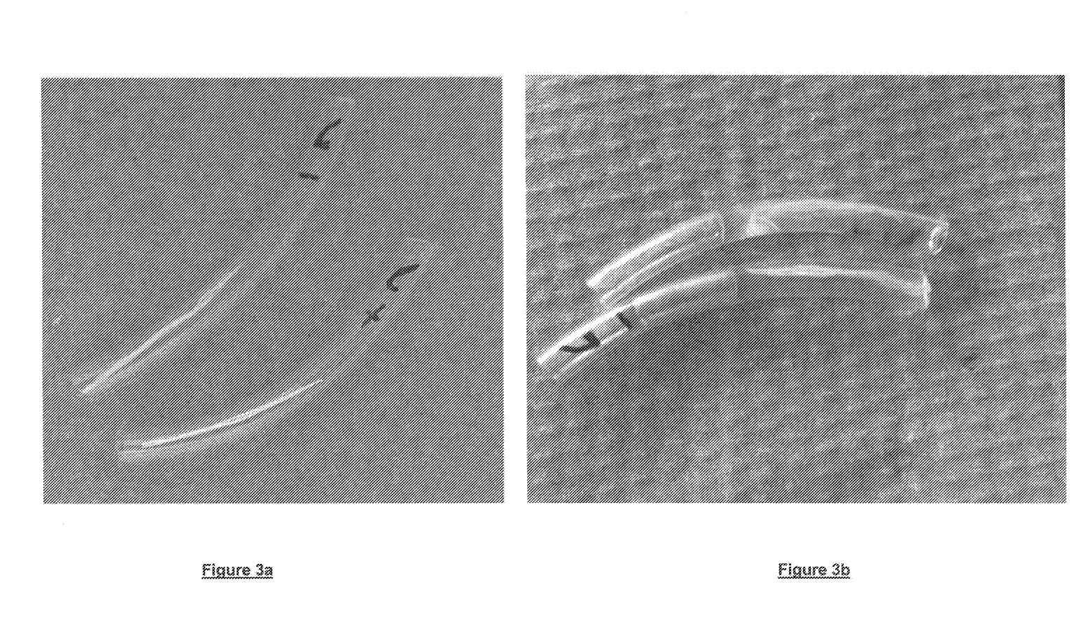

FIG. 3a shows a haemostatic suture and a control suture placed into polypropylene tubes;

FIG. 3b shows polymerisation of plasma by a haemostatic suture;

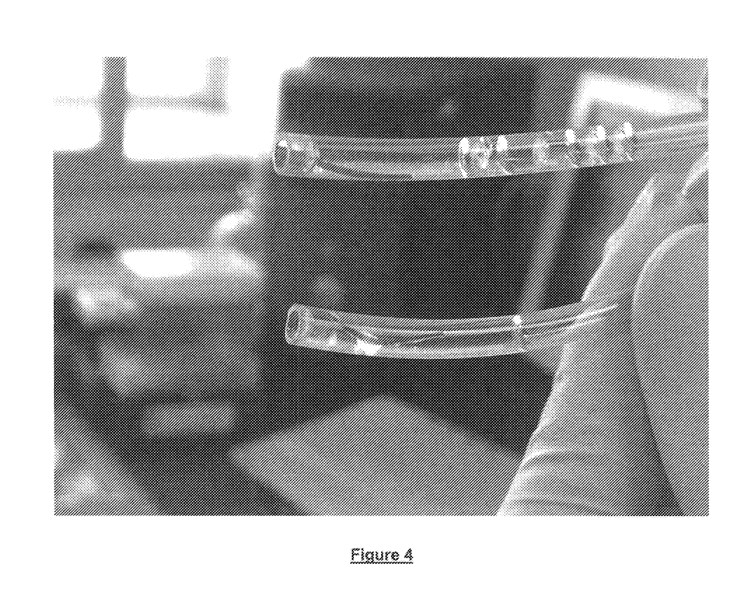

FIG. 4 shows a haemostatic suture forming a clot when contacted with fibrinogen;

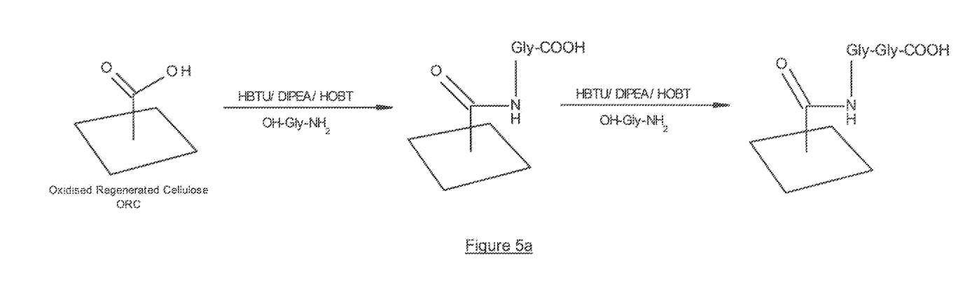

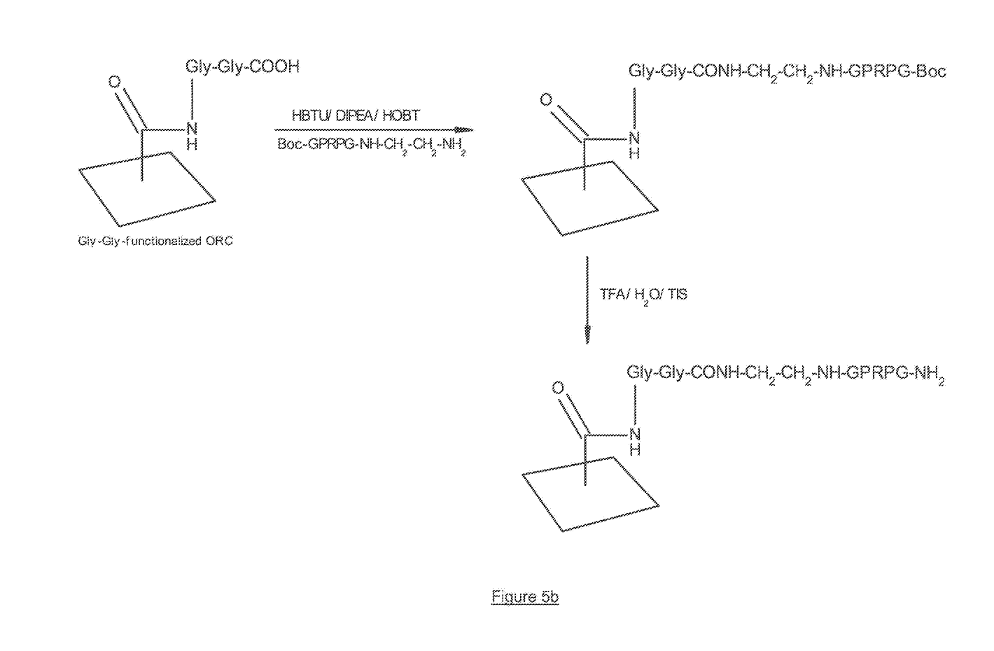

FIG. 5a shows reaction scheme for modifying a surgical fastener;

FIG. 5b shows a reaction scheme for covalently immobilising a fibrinogen binding peptide to a modified surgical fastener;

FIG. 5c shows a positive Kaiser test undertaken on a suture thread having fibrinogen binding peptides covalently immobilised to it;

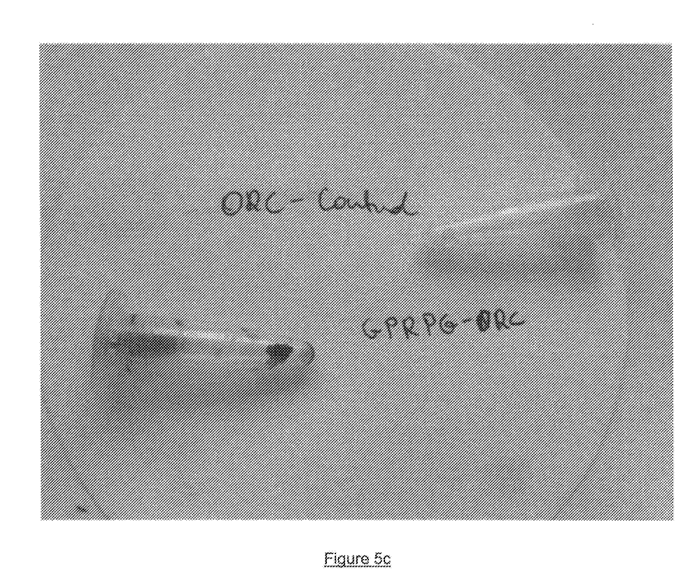

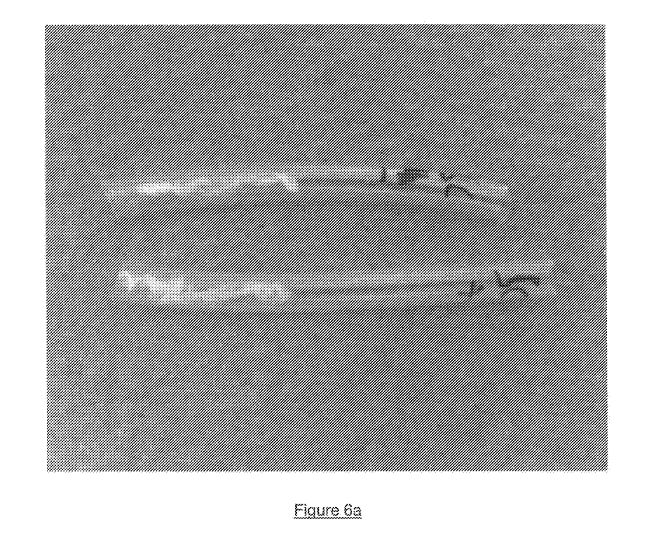

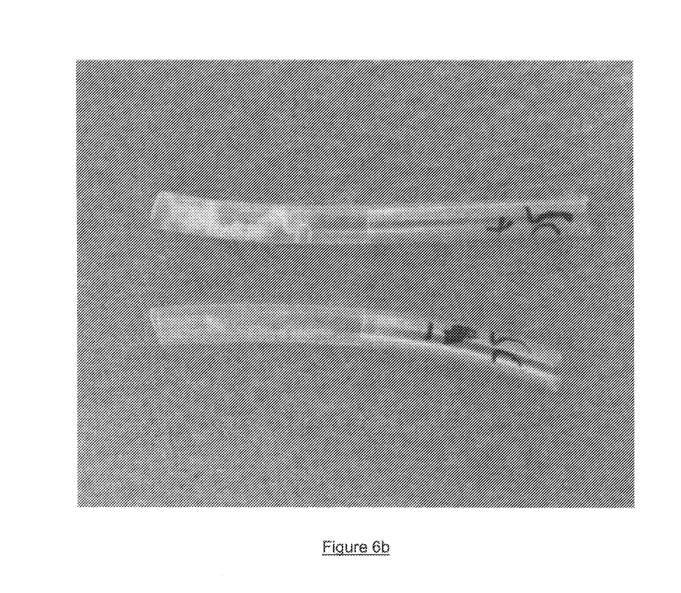

FIG. 6a shows a haemostatic suture and a control suture placed into polypropylene tubes;

FIG. 6b shows polymerisation of fibrinogen in human blood plasma by a haemostatic suture

FIG. 6c shows a fibrinogen clot on a haemostatic suture;

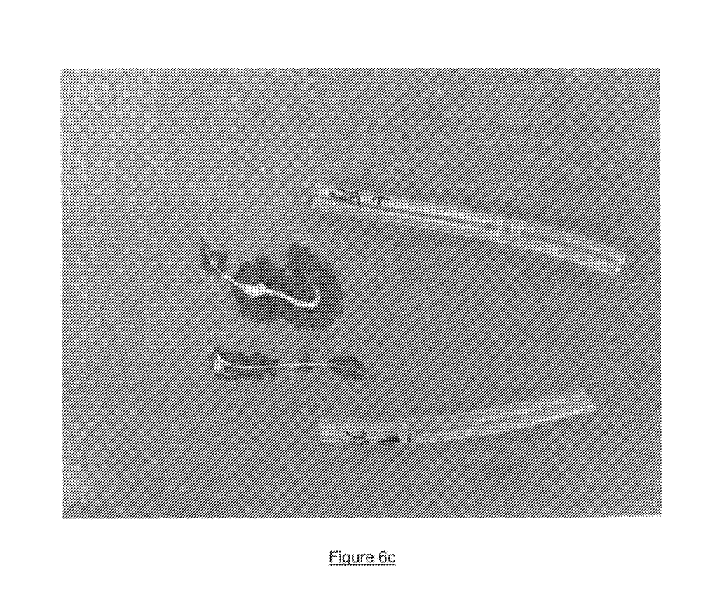

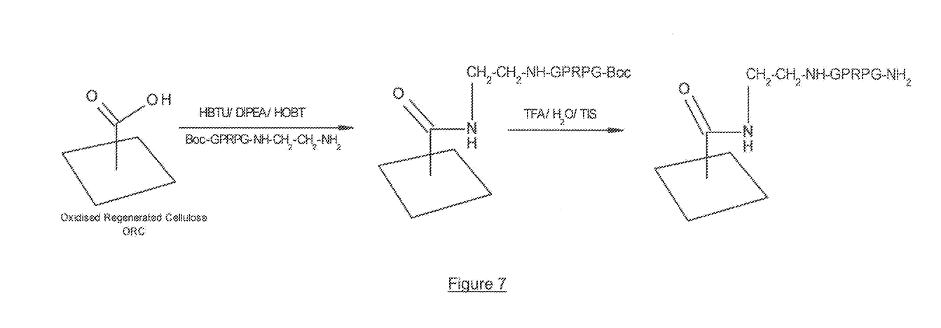

FIG. 7 shows a reaction scheme for covalently immobilising a fibrinogen binding peptide to a surgical fastener;

FIG. 8a shows a reaction scheme for modifying, a surgical fastener;

FIG. 8b shows a reaction scheme for covalently immobilising a fibrinogen binding peptide to a modified surgical fastener;

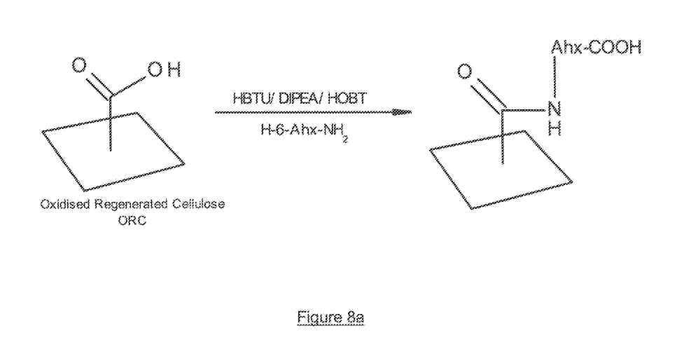

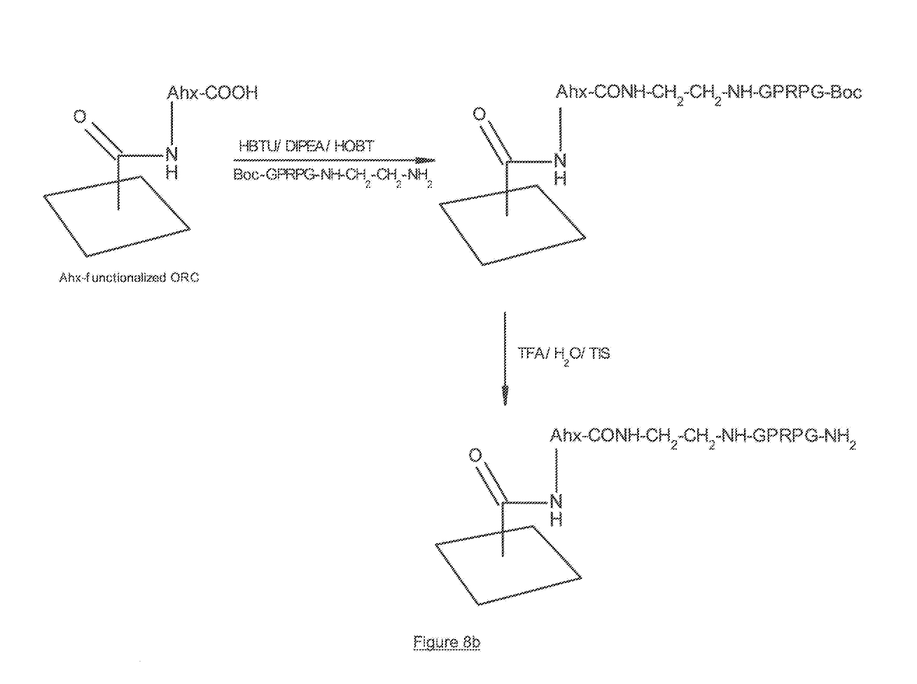

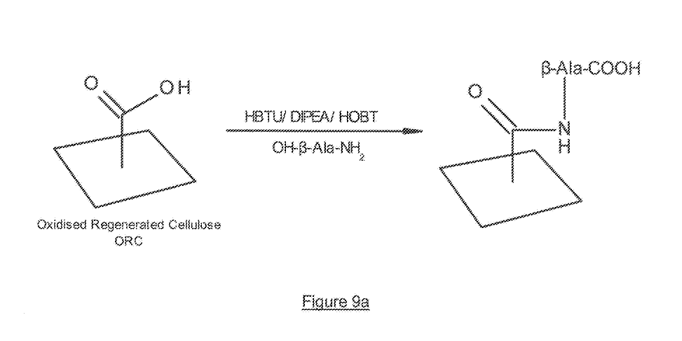

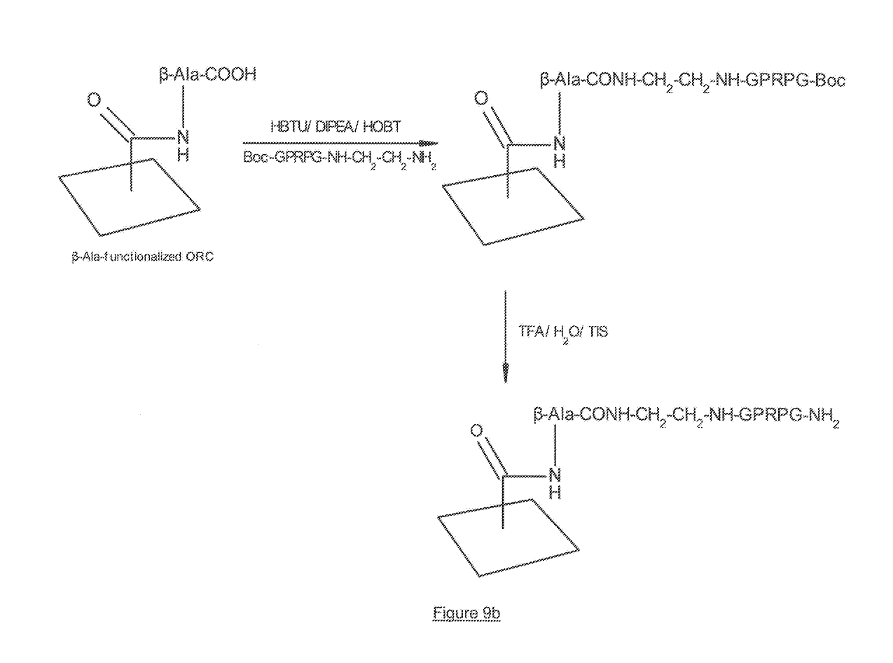

FIG. 9a shows a reaction scheme for modifying a surgical fastener;

FIG. 9b shows a reaction scheme for covalently immobilising a fibrinogen [symbol] to a modified surgical fastener;

FIG. 10 shows the ability of a peptide dendrimer to polymerise fibrinogen at varying concentrations;

FIG. 11 shows the ability of several different peptide dendrimers to polymerise fibrinogen at varying concentrations. The numbering refers to the identity of the peptide dendrimer;

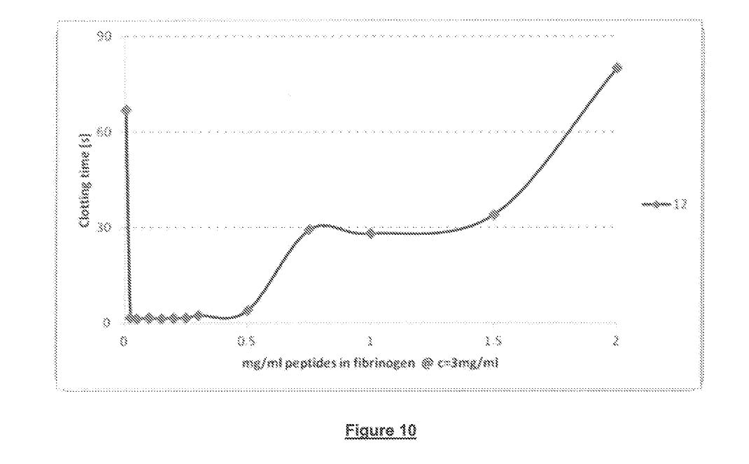

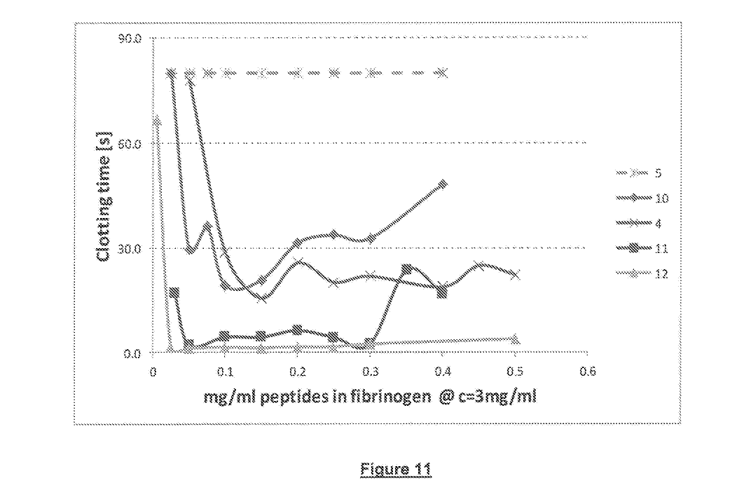

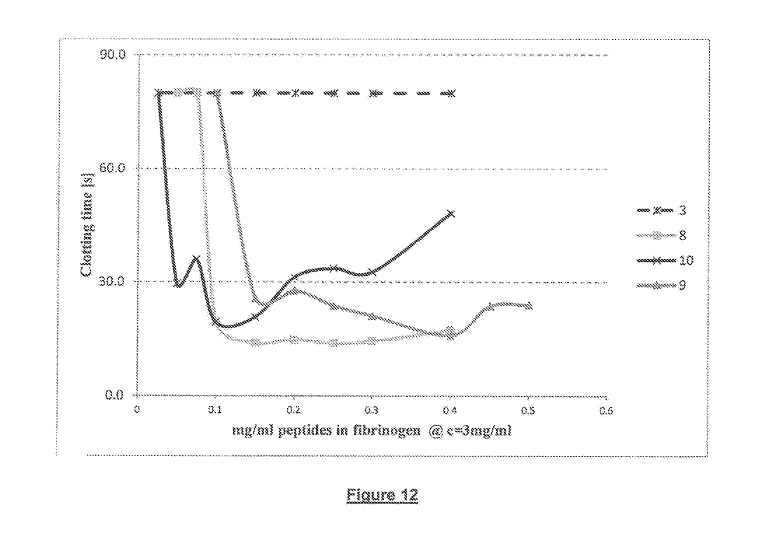

FIG. 12 shows the ability of several different peptide dendrimers to polymerise fibrinogen at varying concentrations. The numbering refers to the identity of the peptide dendrimer;

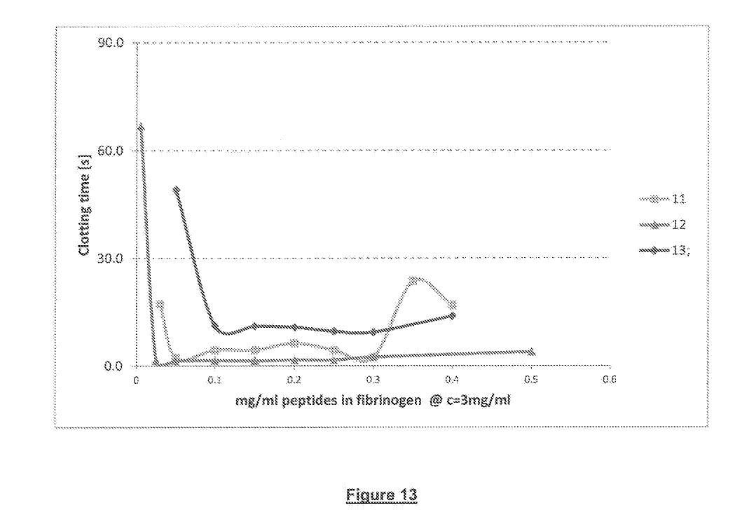

FIG. 13 shows the ability of several different peptide dendrimers to polymerise fibrinogen at varying concentrations. The numbering refers to the identity of the peptide dendrimer;

FIG. 14 shows a photograph of hydrogels formed by polymerisation of fibrinogen using different peptide dendrimers;

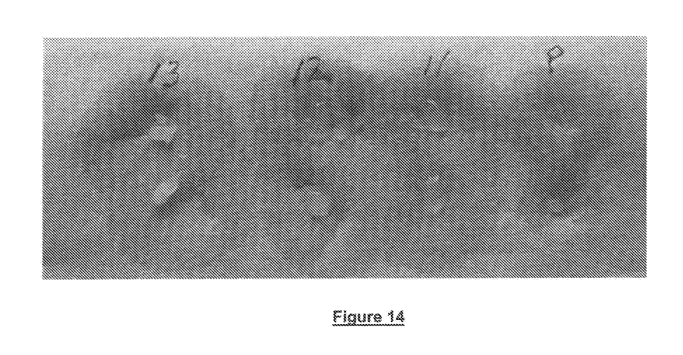

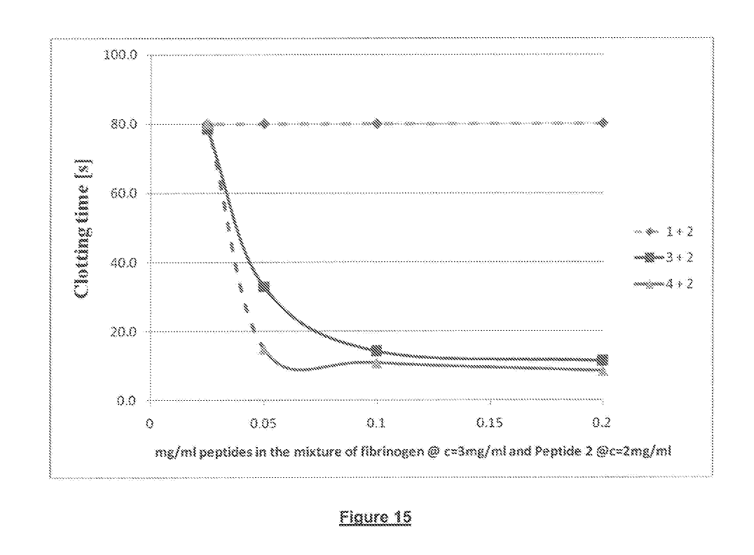

FIG. 15 shows the ability of different combinations of peptide dendrimers with peptide conjugates to polymerise fibrinogen at varying concentrations; and

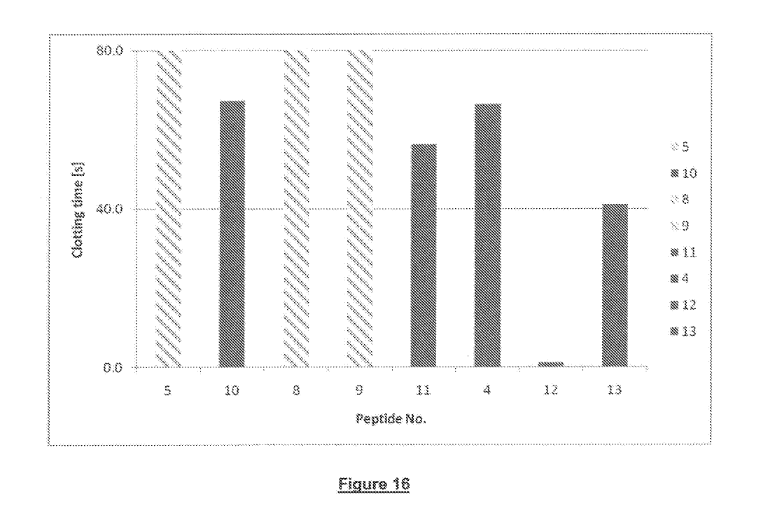

FIG. 16 shows the ability of several different peptide dendrimers to polymerise fibrinogen in human plasma.

### EXAMPLE 1

### Silk Fibres Coated with a Haemostatic Agent (PeproStat)

PeproStat is a haemostatic agent comprising fibrinogen-binding peptides (each having the sequence GPRPG) immobilised to an albumin carrier.

Silk fibres (60 mg) were immersed in a solution of PeproStat (60 μl, 18,6 mg/ml, Batch No RX500552.002 formulated in 20 mM Tris Buffer, 150 mM NaCl; pH=7.2). As a control, silk fibres (60 mg) were placed into Tris buffer (60 μl, 20 mM Tris Buffer, 150 mM NaCl, pH=7.2).

The treated silk fibres were dried overnight at 33° C. then placed into separate polypropylene tubes.

A fibrinogen solution (150 μl, at physiological concentration 3 mg/ml, sourced from Enzyme Research Laboratories Batch No. F1B14230L, formulated in 20 mM Tris buffer, pH=7.2) was added to each sample and the tubes were incubated at 33° C. for 3 minutes.

The results are shown in FIG. 1, with Tube-P (bottom) as the PeproStat c [symbol] sample and Tube-C (top) as the control. FIG. 1 shows the ability of PeproStat-silk fibre to copolymerise with fibrinogen. The results show that silk fibre was not able to form a clot with fibrinogen in the control sample.

### EXAMPLE 2

### Cotton (Gauze ) Fibres Coated with a Haemostatic Agent (Dendrimer P12)

The structure of peptide dendrimer 12 (P12) is shown below, in Example 5.

Cellulose (cotton) fibres were placed into a solution of dendrimer P12 (60 μl, 5 mg/ml, 20 mM Phosphate buffer, pH=7.2) or Phosphate buffer (60 μl, 20 mM Phosphate buffer pH=7.2). The treated fibres were dried for 2 h at 33° C. then placed into separate polypropylene tubes. FIG. 2a shows the fibres placed into polypropylene tubes (Tube P-12 at the top, Tube-C at the bottom).

A fibrinogen solution (150 μl, at physiological concentration 3 mg/ml sourced from Enzyme Research Laboratories Batch No. F1B14230L), formulated in 20 mM Phosphate buffer pH=7.2) was added to each sample and the tubes were incubated at 33° C. for 3 minutes. FIG. 2b illustrates polymerization of fibrinogen (milky gel) with P-12 (Tube-P12 (top)) and control sample (Tube-C (bottom)). FIGS. 2c-2e show the tubes being held vertically at progressive time points, from left to right. A clot in Tube P-12 (left) occluded the tube and prevented dripping. Dripping was not prevented in Tube-C (right) because there was no clot occluding the tube.

The same preparation procedure was followed for cotton-P12 in human plasma. Each fibre was incubated with 150 μl of human plasma at 33° C. for 3 minutes. FIG. 3a shows the cotton fibres placed in polypropylene tubes (Tube-C (top) and Tube P-12 (bottom)). FIG. 3b illustrates that polymerisation of fibrinogen occurred in human plasma (milky gel) in Tube-P12 (Top), but not with the control sample (Tube-C (bottom)).

### EXAMPLE 3

### Heat Immobilisation of a Haemostatic Agent (PeproStat)

10 mm long sutures—(LOT CKE627—Ethicon Prolene) were placed into separate glass vials with PeproStat (100 μl, 18.6 mg/ml, Batch No. RX500552.002, formulated in 20 mM Tris Buffer, 150 mM NaCl, pH=7.2). The samples were sealed and placed in water bath which was at 92° C. This was left to cool down to room temperature overnight (16 hours).

Sutures were remove from the glass vials and transferred to separate polypropylene tubes. The fibrinogen solution (150 μl, at physiological concentration 3 mg/ml, Batch No. F1B14230L), formulated in 20 mM Tris buffer, pH=7.2) was added to each 4 demonstrates the ability of thermal grafted PeproStat-suture (Tube-P (below)) to form a clot with human fibrinogen and a lack of a clot in the deionised water sample (Tube-C(above)).

### EXAMPLE 4

### Covalent Immobilisation of Fibrinogen Binding Peptides to a Suture

The fibrinogen binding properties of fibrinogen binding peptides covalently linked to oxidised regenerated cellulose fibres, was tested.

285 mg of commercially available oxidised regenerated cellulose fibres (Surgicel® Original produced by Ethicon Inc.) were used for the synthesis. Carboxylic acid content in oxidised cellulose fibre was adopted from European patent publication EP 0659440. For example 50 grams of Surgicel Nu-C nit® cloth has 20% carboxylic acid content (0.22 moles of carboxylic acid).

Preparation of Surface-Modified Oxidised Cellulose Fibre by Gly-Gly Spacer

Introduction of a Gly-Gly spacer into the oxidised regenerated cellulose (ORC) fibre was accomplished through base-catalysed HBTU/HOBT amide bound formation. Fibre used in the synthesis was pre-washed with 2×5 ml dichloromethane (DCM) (1 min) and dried at 33° C. After drying, the fibre −285 mg (1.25 mmol —COOH concentration) was immersed in a 5 ml dimethylformamide (DMF) solution and mixed with O-benzotriazole-N,N,N′,N′-tetramethyl-uronium-hexafluoro-phosphate (HBTU; 597 mg, 1.56 mmol), 1-hydroxy-1H-benzotriazole (HOBT; 217 mg, 1.56 mmol) then fibre was activated for 15 mins at room temperature. N,N-Diisopropylethylenediamine (3.14 mmol, 0.505 ml, d=0.798) (or N,N-Diisopropylethylamine-DIPEA) was then added and the resulting solution was reacted for another 15 min. After this, 24 mg, 0.31 mmol of Gly-OH dissolved in Dimethylsulfoxide (DMSO) was added to the reaction mixture. The coupling reaction was carried on at room temperature for 2 hours and 30 minutes.

ORC fibres were washed with DMF (3×5 ml), Methanol (MeOH) (3×5 ml) and with DMF (3×5 ml). The Gly-OH coupling step was repeated and incubated for 30 min at room temperature then washed with DMF (2×5 ml), MeOH (1×5 ml) and with DMF (2×5 ml).

FIG. 5a summarises the reaction scheme and structures, during modification of the oxidised cellulose with a Gly-Gly spacer.

Gly-Gly-functionalised ORC fibre was used for coupling to Boc-GPR(Pbf) PG-NH—CH₂—CH₂—NH₂. Boc-GPR(Pbf) PG-NH—CH₂—CH₂—NH₂ (Boc-FBP) peptides were assembled from the C to N-terminus exclusively by Fmoc-chemistry. During the last synthetic point of the synthesis peptide chain was fully protected with free amine group on the C-termini, and Pbf protection group on Arg. Fully protected peptide was purchased from Almac Ltd.

Synthesis of Boc-FBP on Gly-Gly-Functionalised Fibre

The coupling of the Boc-FBP on the Gly-Gly-functionalised fibre was accomplished by a novel adaptation of SPOT synthesis (Hilpert K., Winkler, D., Hancock R.; Nature Protocols; 2007, vol. 2, No. 6, p 1333-1349).

Gly-Gly-functionalized fibre was immersed in a DMF (5 ml) and mixed with HBTU (475 mg, 1.25 mmol), HOBT (169 mg, 1.25 mmol). After stirring at room temperature for 2 min, N,N-Diisopropylethylenediamine (0.406 ml, 2.5 mmol) (or DIPEA) was added and mixed for 2 min. 275 mg (0.31 mmol) of Boc-FBP peptide was dissolved in DMF (200 μl) and this was added to the reaction mixture. The coupling reaction was carried out overnight (17 hours) at room temperature. The fibre then was washed with DMF (3×5 ml) and with DCM (3×5 ml). Removal of protecting groups with 95% TFA, 2.5% TIS, 2.5% water (3 ml) after coupling reaction produced GPRPG-NH—CH₂—CH₂—NH—CO-G-G-fibre (“GPRPG-G-G-ORC”).

FIG. 5b summarises the reaction scheme and structures involved in coupling Boc-FBP to the Gly-Gly functionalised fibre.

The Kaiser test (Ninhydrin test) was used to monitor presence of fully deprotected peptide GPRPG-linker-ORC reminds bound on the cellulose fibre (See FIG, 5c—ORC (Control) (top), GPRPG-G-G-ORC (bottom)).

Functionality Test

FIG. 6a illustrates GPRPG-G-G-ORC (tube labelled SC+) and ORC (control) (tube labelled SG-) fibres placed into separate polypropylene tubes. 150 μl of Human Plasma solution (Alpha Labs-Plasma Lot #A1162 Exp 2016-03) was added to each sample and the fibres were incubated at 37° C. for 1.5 minutes. There is a clot present in SC+(top)—shown in FIG. 6b.

Visual examination of threads removed from the polyethylene tubes was als [symbol] FIG. 6c shows that GPRPG-G-G-ORG fibre formed a clot with human fibrinogen. The GPRPG-G-G-ORC fibre removed from the container was thicker than the control sample.

Samples of GPRPG-G-G-FBP and ORC (control) were weighed out and treated with 150 μl of human plasma (Alpha Labs-Plasma Lot #A1174 Exp 2016-03) and incubated for 1.5 min at 33° C. Tested samples and controls were removed from the plasma and then were weighed to determine if any difference was observable. The test was repeated four times. The results in Table 1 show that the mass remaining on GPRPG-G-G-ORC was significantly higher compared to control samples, suggesting that fibrinogen-binding peptides retain activity when conjugated to the regenerated oxidised cellulose material.

TABLE 1

| | | | Incubation time with |
|---|---|---|---|
| | Starting mass | End mass | 150 μl of human |
| Tested samples | (mg) | (mg) | plasma (min) |
| ORC (control) | 8 | 65 | 1.5 |
| GPRPG-G-G-ORC | 8 | 89 | 1.5 |
| ORC (control) | 9 | 50 | 1.5 |
| GPRPG-G-G-ORC | 9 | 83 | 1.5 |
| ORC (control) | 6 | 43 | 1.5 |
| GPRPG-G-G-ORC | 7 | 50 | 1.5 |
| ORC (control) | 10 | 78 | 1.5 |
| GPRPG-G-G-FBP | 10 | 85 | 1.5 |

One Step Coupling of Boc-GPR (Pbf) PG-NH—CH₂—CH₂—NH₂ (Boc-F8P-) on oxidised regenerated cellulose material.

Bor-GPR (Pbf) PG-NH—CH₂—CH₂—NH₂ (Boc-FBP-) moieties were assembled from the C to N terminus exclusively by Fmoc-chemistry. During the last synthetic point of the synthesis, the moieties were fully protected with free amine group on the C-termini including a Pbf protection group on Arg. Protected moieties were purchased from Almac Ltd. Commercially available Surgicel* Original Absorbable Hemostat (oxidised regenerated cellulose (ORC)) made by Ethicon Inc. of Johnson & Johnson Medical Limited was used as the substrate. Carboxylic acid content in Surgicel was adopted from the literature (See EP 0659440). 50 grams of Surgical® Nu Knit®* cloth has 20% carboxylic acid [symbol] moles of carboxylic acid).

ORC material used in the synthesis was pre-washed with 2×1 ml dichloromethane (DCM) (1 min) and dried at 33° C. After drying, the ORC material −50 mg (0.2 mmol—of carboxylic acid COOH) was immersed in a 1 ml dimethylformamide (DMF) solution and mixed with O-benzotriazole-N,N,N′,N′-tetramethyl-uronium-hexafluoro-phosphate (HBTU; 90 mg, 0.2 mmol), 1-hydroxy-1H-benzotriazole (HOBT; 30 mg, 0.2 mmol) then dressing was activated for 15 min at room temperature. N,N-Diisopropylethylenediamine (0.4 mmol, 0.075 ml, d=0.798) (or N,N-Diisopropylethylamine DIPEA) was then added and resulting solution reacted for another 15 min. After this, 50 mg, 0.05 mmol of Boc-GPR (Pbf) PG-NH—CH₂—CH₂—NH₂ dissolved in DMF was added to the reaction mixture—2 ml in total. The coupling reaction was carried on at room temperature for 5 hours. The material was washed with DMF (3×1 ml), Methanol (MeOH) (3×1 ml) and with DMF (3×1 ml). The Boc-GPR (Pbf) PG-NH—CH₂—CH₂—NH₂ coupling step was repeated and incubated overnight at room temperature then washed with DMF (2×1 ml), MeOH (1×1 ml) and with DMF (2×1 ml). The ORC material was then washed with DMF (3×5 ml) and with DCM (3×5 ml). Removal of protecting groups with 95% TFA, 2.5% TIS, 2.5% water (3 ml) after the coupling reaction produced GPRPG-NH—CH₂—CH₂—NH—CO-ORC (“GPRPG-ORC”).

FIG. 7 summarises the reaction scheme and structures.

Functionality Test

Samples of GPRPG-FBP and ORC (control) were weighed out, treated with 100 μl of human plasma (Alpha Labs-Plasma Lot #A1162 Exp 2015-03) and incubated for 1.5 or 3 min at 33° C. Tested samples and controls were removed from the plasma and then were weighed to determine any difference, The test was repeated 3 times. The results in Table 2 show that the mass remaining on the GPRPG-ORC is significantly higher compared to control samples, indicating that fibrinogen binding peptides retain activity when conjugated to the material.

TABLE 2

| | | | Incubation time with |
|---|---|---|---|
| | Starting mass | End mass | 100 μl of human |
| Tested samples | (mg) | (mg) | plasma (min) |
| ORC (control) | 6 | 42 | 3 |
| GPRPG-ORC | 6 | 66 | 3 |
| ORC (control) | 5 | 21 | 1.5 |
| GPRPG-ORC | 5 | 45 | 1.5 |
| ORC (control) | 3 | 27 | 1.5 |
| GPRPG-ORC | 3 | 42 | 1.5 |

Preparation of Surface-Modified Oxidised Cellulose Material with ε-Ahx Spacer

Introduction of 6-aminohexanoic acid (ε-Ahx) spacer into the oxidised cellulose material was accomplished through base catalysed HBTU/HOBT amide bond formation. The synthetic method employed was substantially the same as described for modification of ORC material with Gly-Gly spacers.

After prewashing and drying steps, 114 mg of ORC material was immersed in 2 ml of DMF solution and mixed with O-benzotriazole-N,N,N′,N′-tetramethyl-uronium-hexafluoro-phosphate (HBTU; 237 mg, 0.625 mmol), 1-hydroxy-1H-benzotriazole (HOBT; 84 mg, 0.625 mmol) then the material was activated for 15 min at room temperature. N,N-Diisopropylethylenediamine (1.25 mmol, 0.200 ml, d=0.798) (or DIPEA) was then added and resulting solution reacted for another 15 min. After this, 16.4 mg, 0.125 mmol of ε-Ahx-OH dissolved in Dimethylsulfoxide (DMSO) was added to the reaction mixture. The coupling reaction was carried out at room temperature overnight.

The material was washed with DMF (3×3 ml), Methanol (MeOH) (3×3 ml) and with DMF (3×3 ml).

FIG. 8a summarises the reaction scheme and the structures.

Coupling of Boc-FBP to the ε-Ahx-Functionalised Dressing

A summary of the reaction scheme, and the structures, is shown in FIG. 8

[symbol]

Firstly, ε-Ahx-functionalised dressing was immersed in a DMF (2 ml) and mixed with HBTU (190 mg, 0.5 mmol), HOBT (67.4 mg, 0.5 mmol). After stirring at room temperature for 2 min N,N-Diisopropylethylenediamine (0.180 ml, 1.1 mmol) (or DIPEA) was added and mixed for 2 min. 110 mg (0.125 mmol) of Boc-FBP peptide was dissolved in DMF (200 μl) and this was added to the reaction mixture. The coupling reaction was carried out overnight (17 hours) at room temperature. The dressing then was washed with DMF (3×3 ml) and with DCM (3×3 ml). Removal of protecting groups with 95% TFA, 2.5% TIS, 2.5% water (3 ml) after the coupling reaction produced GPRPG-NH—CH₂—CH₂—NH—CO-Ahx-ORC (“GPRPG-Ahx-ORC”).

The Kaiser Test (Ninhydrin test) test was used to monitor presence of fully deprotected peptide remaining bound on the cellulose.

Preparation of Surface-Modified Oxidised Cellulose Material with β-Ala Spacer

Introduction of β-alanine (β-Ala) spacer into the oxidised cellulose material was accomplished through base catalysed HBTU/HOBT amide bond formation.

After prewashing and drying steps, 206 mg of material was immersed in 5 ml of DMF solution and mixed with O-benzotriazole-N,N,N′,N′-tetramethyl-uronium-hexafluoro-phosphate (HBTU; 442 mg, 1.165 mmol), 1-hydroxy-1H-benzotriazole (HOBT; 152 mg, 1.125 mmol) then the material was activated for 15 min at room temperature. N,N′-Diisopropylethylenediamine (2.468 mmol, 0.319 ml) (or N,N-Diisopropylethylamine-DIPEA) was then added and resulting solution reacted for another 15 min. After this, 20 mg, 0.226 mmol of (β-Ala-OH dissolved in Dimethylsulfoxide (DMSO) was added to the reaction mixture. The coupling reaction was carried out at room temperature overnight.

The material was washed with DMF (3×5 ml), Methanol (MeOH) (3×5 ml) and with DMF (3×5 ml).

FIG. 9a summarises the reaction scheme and the structures.

Coupling of Boc-FBP to the (β-Ala-Functionalised Dressing The coupling of the Boc-FBP to the β-Ala-functionalised material was accon [symbol] base catalysed synthesis approach.

A summary of the reaction scheme, and the structures, is shown in FIG. 9b.

Firstly, β-Ala-functionalised dressing was immersed in a DMF (5 ml) and mixed with HBTU (350 mg, 0.923 mmol), HOBT (124 mg, 0.918 mmol). After stirring at room temperature for 2 min N,N′-Diisopropylethylenediamine (0.247 ml, 1.911 mmol) (or N,N-Diisopropylethylamine DIPEA) was added and mixed for 2 min. 202 mg (0.231 mmol) of Boc-FBP peptide was dissolved in DMF (400 μl) and this was added to the reaction mixture. The coupling reaction was carried out overnight (17 hours) at room temperature. The dressing then was washed with DMF (3×5 ml) and with DCM (3×5 ml). Removal of protecting groups with 95% TFA, 2.5% TIS, 2.5% water (3 ml) after the coupling reaction produced GPRPG-NH—CH₂—CH₂—NH—CO-β-Ala-ORC (“GPRPG-β-Ala-ORC”).

The Kaiser Test was used to monitor the presence of fully deprotecte.d peptide remaining bound on the cellulose.

Functionality Test

Samples of GPRPG-β-Ala-FBP, GPRPG-Ahx-ORC and ORC (control) were weighed out and treated with 100 μl of human plasma (Alpha Labs-Plasma Lot #A1174 Exp 2016-03) and incubated for 1.5 min at 33° C. Tested samples and controls were removed from the plasma and then were weighed to determine if any difference was observable. The test was repeated three times. The results in Table 3 showed that the mass remaining on GPRPG-β-Ala-ORC, GPRPG-Ahx-ORC was significantly higher compared to control (Surgicel) samples, suggesting that fibrinogen-binding peptides retain activity when conjugated to the regenerated oxidised cellulose material.

TABLE 3

| | | | Incubation time with |
|---|---|---|---|
| | Starting mass | End mass | 100 μl of human |
| Tested samples | (mg) | (mg) | plasma (min) |
| ORC (control) | 4 | 34 | 1.5 |
| GPRPG-β-Ala-FBP | 4 | 64 | 1.5 |
| GPRPG-Ahx-ORC | 4 | 54 | 1.5 |
| ORC (control) | 4 | 36 | 1.5 |
| GPRPG-β-Ala-FBP | 4 | 60 | 1.5 |
| GPRPG-Ahx-ORC | 4 | 50 | 1.5 |
| ORC (control) | 4 | 32 | 1.5 |
| GPRPG-β-Ala-FBP | 4 | 59 | 1.5 |
| GPRPG-Ahx-ORC | 4 | 57 | 1.5 |

### EXAMPLE 5

### Synthesis of Peptide Dendrimers and Peptide Conjugates

Peptides were synthesised on Rink amide MBHA low loaded resin (Novabiochem, 0.36 mmol/g), by standard Fmoc peptide synthesis, using Fmoc protected amino acids (Novabiochem).

In general, single-coupling cycles were used throughout the synthesis and HBTU activation chemistry was employed (HBTU and PyBOP (from AGTC Bioproducts) were used as the coupling agents). However, at some positions coupling was less efficient than expected and double couplings were required.

The peptides were assembled using an automated peptide synthesiser and HBTU up to the branch points and by manual peptide synthesis using PyBOP for the peptide branches.

For automated synthesis a threefold excess of amino acid and HBTU was used for each coupling and a ninefold excess of N,N-Diisopropylethylamine (DIPEA, Sigma) in dimethylformamide (DMF, Sigma).

For manual synthesis a threefold excess of amino acid and PyBOP was used for each coupling and a ninefold excess of DIPEA in N-methylpyrollidinone (NMP, Sigma).

Deprotection (Fmoc group removal) of the growing peptide chain using 20% piperidine (Sigma) in DMF likewise may not always be efficient and require double deprotection.

Branches were made using Fmoc-Lys(Fmoc)-OH, Fmoc-Lys(Boc)-OH, or F [symbol] OH.

Final deprotection and cleavage of the peptide from the solid support was performed by treatment of the resin with 95% TFA (Sigma) containing triisopropylsilane (TIS, Sigma), water and anisole (Sigma) (1:1:1, 5%) for 2-3 hours.

The cleaved peptide was precipitated in cold diethyl ether (Sigma) pelleted by centrifugation and lyophilized. The pellet was re-dissolved in water (10-15 mL), filtered and purified via reverse phase HPLC using a C-18 column (Phenomenex at flow rate 20 ml/min) and an acetonitrile/water gradient containing 0.1% TFA. The purified product was lyophilized and analyzed by ESI-LC/MS and analytical HPLC and were demonstrated to be pure (>95%). Mass results all agreed with calculated values.

Peptide Dendrimers and Peptide Conjugates

The structures of peptide dendrimers and peptide conjugates synthesised using the methods described above are shown below

The “NH₂—” group at the end of a peptide sequence denotes an amino group at the amino-terminal end of the sequence. The “-am” group at the end of a peptide sequence denotes an amide group at the carboxy-terminal end of the sequence.

Peptide Conjugate No: 1:

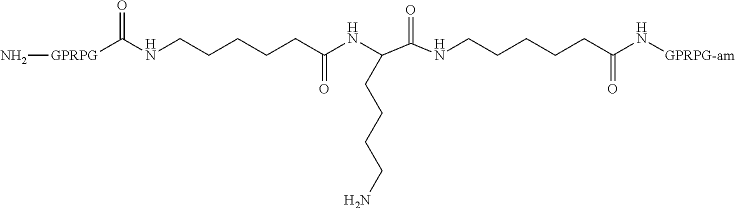

Peptide Conjugate No, 2:

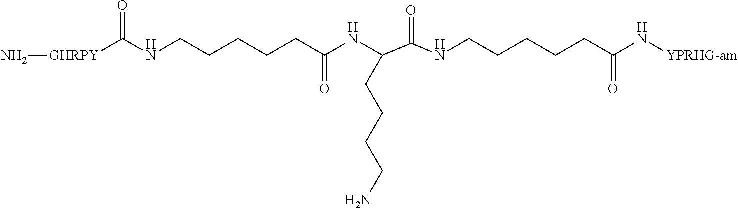

Peptide Dendrimer No. 3:

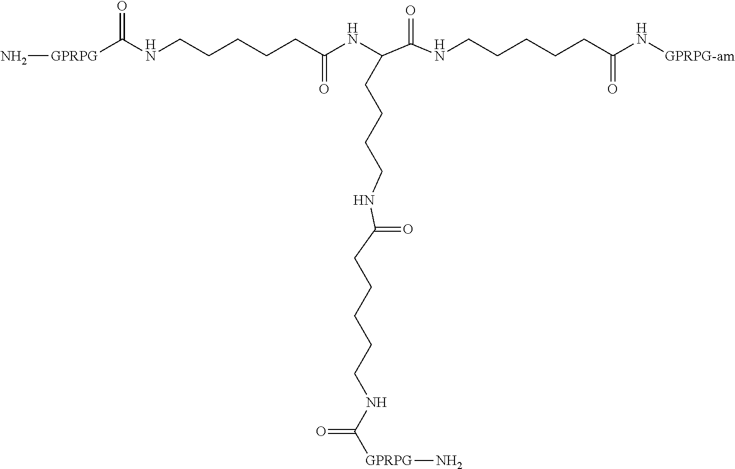

Peptide Dendrimer No. 4:

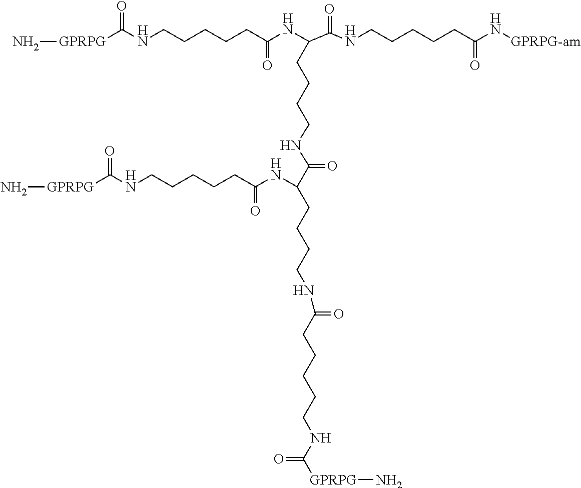

Peptide Dendrimer No. 5:

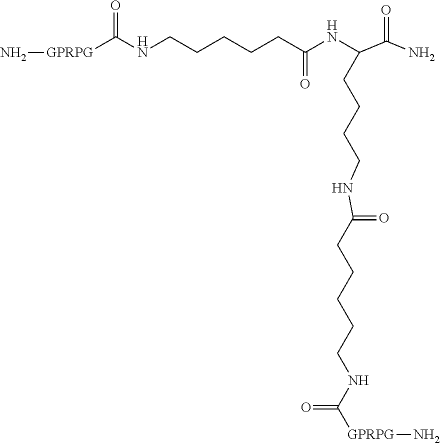

Peptide Dendrimer No. 8:

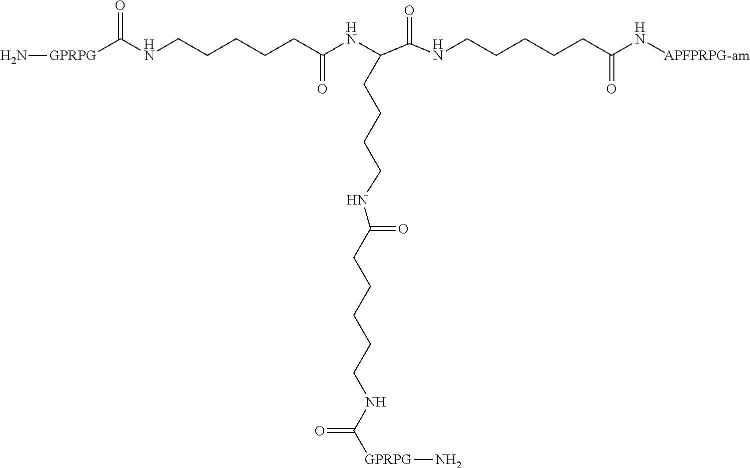

Peptide Dendrimer No. 9:

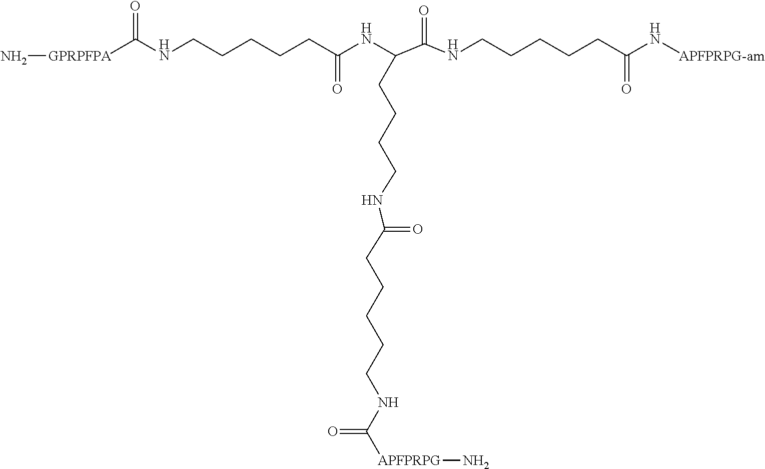

Peptide Dendrimer No. 10:

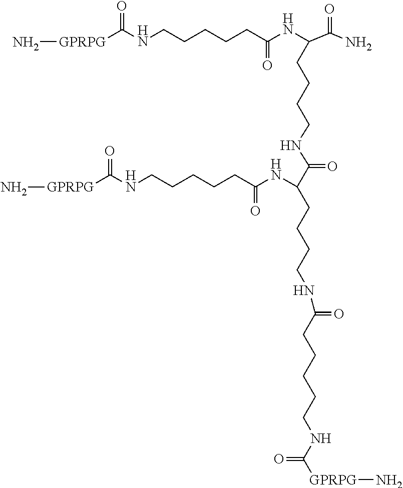

Peptide Dendrimer No. 11:

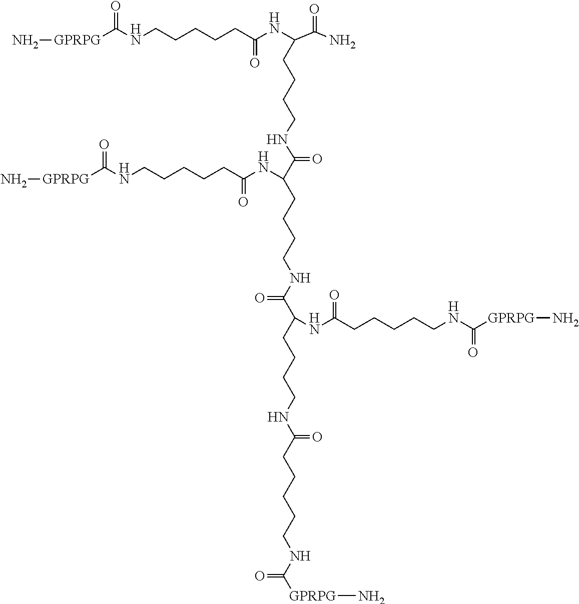

Peptide Dendrimer No. 12:

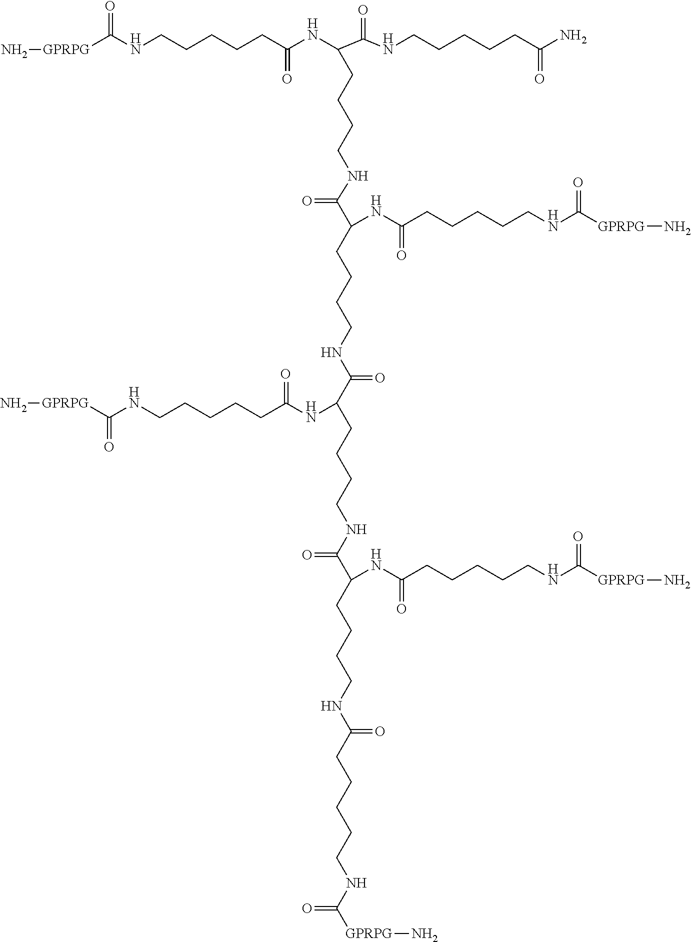

Peptide Dendrimer No. 13:

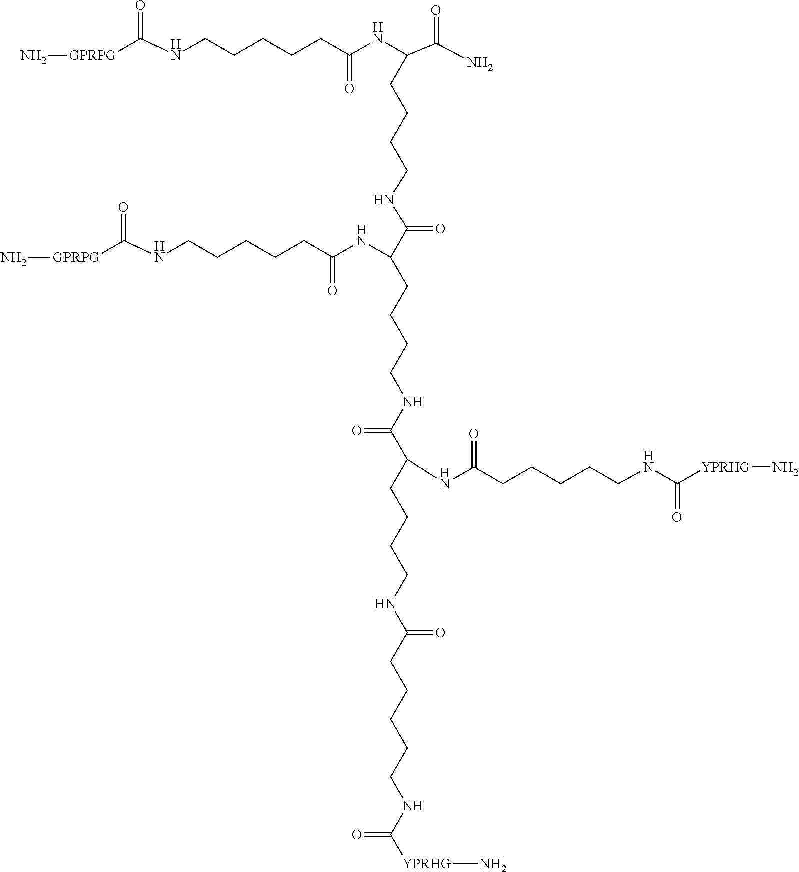

### EXAMPLE 6

### Co-Polymerisation of a Peptide Dendrimer with Fibrinogen

Dendrimer No. 12 comprises a branched core with five consecutive lysine residues. The lysine residues are covalently linked through a side chain of an adjacent lysine, residue.

The ability of Peptide Dendrimer No. 12 to polymerise fibrinogen was assessed. 30 μl of dendrimer in solution, at concentration ranging from 0.005-2 mg/ml, was added to 100 μl purified human fibrinogen at 3 mg/ml (the level of fibrinogen found in the blood). Polymerisation of fibrinogen was analysed using a Sigma Amelung KC4 Delta coagulation analyser. FIG. 10 shows a plot of the polymerisation (clotting) times (in se [symbol] increasing concentration of dendrimer.

The results show that the dendrimer was able to copolymerise with fibrinogen almost instantaneously, even at very low concentrations of dendrimer. The increase in clotting time with dendrimer concentrations above 0.5 mg/ml is thought to be explained by an excess of fibrinogen-binding peptides compared to the number of free binding pockets in fibrinogen. At higher concentrations, the fibrinogen-binding peptides of the dendrimer may saturate the fibrinogen binding pockets, resulting in a significant number of excess dendrimer molecules that are not able to copolymerise with fibrinogen.

### EXAMPLE 7

### Effect of Varying the Number of Fibrinogen-Binding Peptides Per Dendrimer on the Speed of Copolymerisation with Fibrinogen

This example investigates the effect of varying the number of fibrinogen-binding peptides per peptide dendrimer on the speed of copolymerisation with fibrinogen.

The ability of Peptide Dendrimer Nos. 4, 5, 10, 11, and 12 to copolymerise with fibrinogen was assessed using the same method described in Example 6. The concentration of each dendrimer was varied from 0.005-0.5 mg/ml. FIG. 11 shows a plot of the clotting times (in seconds) with increasing concentration of each different dendrimer.

The results show that dendrimer No. 5 (with only two fibrinogen-binding peptides/dendrimer) was not able to copolymerise with fibrinogen. As the number of fibrinogen-binding peptides was increased from three to five, at concentrations of dendrimer from ˜0.125 to ˜0.275 mg/ml, the speed of cc polymerisation increased. At concentrations below ˜0.125 mg/ml dendrimer, dendrimer No. 10 (with three fibrinogen-binding peptides/dendrimer) produced faster clotting times than dendrimer no. 4 (with four fibrinogen-binding peptides/dendrimer). In the range, ˜0.02-0.5 mg/ml, dendrimer no. 12 (with five fibrinogen-binding peptides/dendrimer) produced almost instantaneous clotting. In the range ˜0.05-0.3 mg/ml, dendrimer no. 11 (with four fibrinogen-binding peptides/dendrimer) also produced almost instantaneous clotting.

It is concluded that the speed at which fibrinogen is polymerised by a dendrimer of the invention generally increases as the number of fibrinogen-binding peptides per dendrimer is increased.

### EXAMPLE 8

### Effect of Fibrinogen-Binding Peptide Orientation, and of Different Fibrinogen-Binding Peptide Sequences on Speed of Copolymerisation with Fibrinogen

To assess whether the orientation of a fibrinogen-binding peptide could affe [symbol] a peptide dendrimer to copolymerise with fibrinogen, peptide dendrimers comprising three fibrinogen-binding peptides attached to a single tri-functional amino acid residue (lysine) were synthesised (referred to as ‘three-branch’ dendrimers), but with one of the fibrinogen-binding peptides orientated with its amino-terminal end attached to the branched core, and amidated at its carboxy-terminal end. The ability of peptide dendrimers comprising different fibrinogen-binding peptide sequences to copolymerise with fibrinogen was also tested.

The fibrinogen-binding peptides of Peptide Dendrimer Nos. 3 and 10 are each of sequence GPRPG (SEQ ID NO: 18). Each fibrinogen-binding peptide of Peptide Dendrimer No. 10 is orientated with its carboxy-terminal end attached to the branched core. One of the fibrinogen-binding peptides of Peptide Dendrimer No. 3 is orientated with its amino-terminal end attached to the branched core. The carboxy-terminal end of that peptide comprises an amide group.

Two of the fibrinogen-binding peptides of Peptide Dendrimer No. 8 are of sequence GPRPG (SEQ ID NO: 18), and the third fibrinogen-binding peptide is of sequence APFPRPG (SEQ ID NO: 2) orientated with its amino-terminal end attached to the branched core. The carboxy-terminal end of that peptide comprises an amide group.

Two of the fibrinogen-binding peptides of Peptide Dendrimer No. 9 are of sequence GPRPFPA (SEQ ID NO: 7), and the third fibrinogen-binding peptide is of sequence APFPRPG (SEQ ID NO: 2) orientated with its amino-terminal end attached to the branched core. The carboxy-terminal end of that peptide comprises an amide group.

The sequence GPRPG (SEQ ID NO: 18) binds to hole ‘a’ and hole ‘b’ of fibrinogen, but with some preference for hole ‘a’. The sequence GPRPFPA (SEQ ID NO: 7) binds with high preference for hole ‘a’ in fibrinogen. The sequence Pro-Phe-Pro stabilizes the backbone of the peptide chain and enhances the affinity of the knob-hole interaction (Stabenfeld et al., BLOOD, 2010, 116: 1352-1359).

The ability of the dendrimers to copolymerise with fibrinogen was assessed using the same method described in Example 6, for a concentration of each dendrimer ranging from 0.005-0.5mg/ml. FIG. 12 shows a plot of the clotting times (in seconds) obtained with increasing concentration of each different dendrimer.

The results show that changing the orientation of one of the fibrinogen-binding peptides of a three-branch dendrimer, so that the peptide is orientated with its amino-terminal end attached to the branched core (i.e. Dendrimer No. 3), reduced the ability of the dendrimer to copolymerise with fibrinogen (compare the clotting time of Dendrimer No. [symbol] Dendrimer No. 10). However, at higher fibrinogen concentrations, Dendrimer No. 3 was able to copolymerise with fibrinogen (data not shown).

A three-branch dendrimer with a fibrinogen-binding peptide of different sequence orientated with its amino-terminal end attached to the branched core was able to copolymerise with fibrinogen (see the results for Dendrimer No. 8).

A three-branch dendrimer in which two of the fibrinogen-binding peptides comprise sequence that binds preferentially to hole ‘b’ in fibrinogen (sequence GPRPFPA (SEQ ID NO: 7)), with these peptides orientated with their carboxy-terminal end attached to the branched core, and the other peptide comprising the reverse sequence (i.e. sequence APFPRPG (SEQ ID NO: 2)) orientated with its amino-terminal end attached to the branched core (Dendrimer No. 9) was also very active in copolymerising with fibrinogen.

### EXAMPLE 9

### Ability of Peptide Dendrimers with Different Fibrinogen-Binding Peptide Sequences to Copolymerise with Fibrinogen

The GPRPG (SEQ ID NO: 18) and GPRPFPA (SEQ ID NO: 7) motifs primarily bind to the ‘a’ hole on fibrinogen. This example describes an assessment of the ability of a chimeric peptide dendrimer (i.e. a peptide dendrimer with different fibrinogen-binding peptide sequences attached to the same branched core) to copolymerise with fibrinogen.

Peptide dendrimer No. 13 is a chimeric four-branch peptide dendrimer' comprising two fibrinogen-binding peptides with sequence GPRPG-(SEQ ID NO: 18) (which has a binding preference for the ‘a’ hole), and two fibrinogen-binding peptides with sequence GHRPY-(SEQ ID NO: 15) (which binds preferentially to the ‘b’ hole). Non-chimeric peptide dendrimers Nos. 11 and 12 are four- and five-arm peptide dendrimers, respectively. Each fibrinogen-binding peptide of these dendrimers has the sequence GPRPG-(SEQ ID NO: 18). Each fibrinogen-binding peptide of Dendrimers Nos. 11, 12, and 13 is attached at its carboxy-terminal end to the branched core.

The ability of the dendrimers to copolymerise with fibrinogen was assessed using the same method described in Example 6, for a concentration of each dendrimer ranging from 0.005-0.5 mg/ml. FIG. 13 shows a plot of the clotting times (in seconds) obtained with increasing concentration of each different dendrimer.

The results show that the clotting speed using the chimeric dendrimer was slower than the non-chimeric dendrimers at concentrations below 0.3 mg/ml. However, FIG. 14 shows a photograph of the hydrogels obtained using the different dendrimers. The g [symbol] with the number of the peptide dendrimer used (11, 12, and 13), and “P” labels a hydrogel formed using a product in which several fibrinogen-binding peptides are attached to soluble human serum albumin. The hydrogel formed by the chimeric dendrimer was more dense and contained less fluid compared to the hydrogels formed using dendrimers Nos. 11 and 12 (at 3 mg/ml fibrinogen, or at higher concentrations of fibrinogen). Thus, although the clotting time was slower using the chimeric dendrimer, the hydrogel formed using this dendrimer was more dense.

### EXAMPLE 10

### Ability of Mixtures of Peptide Deridrimers and Peptide Conjugates to Copolymerise with Fibrinogen

Fibrinogen-binding peptide of sequence GPRP-(SEQ ID NO: 6) binds strongly and preferentially to the ‘a’ hole of fibrinogen (Laudano et al., 1978 PNAS 7S). Peptide Conjugate No. 1 comprises two fibrinogen-binding peptides with this sequence, each attached to a lysine residue. The first peptide is attached its carboxy-terminal end by a linker to the lysine residue, and the second peptide is attached at its amino-terminal end by a linker to the lysine residue. The carboxy-terminal end of the second peptide comprises an amide group.

Fibrinogen-binding peptide of sequence GFIRPY-(SEQ ID NO: 15) binds strongly and preferentially to the ‘b’ hole of fibrinogen (Doolittle and Pandi, Biochemistry 2006, 45, 2657-2667). Peptide Conjugate No. 2 comprises a first fibrinogen-binding peptide with this sequence, attached at its carboxy-terminal end by a linker to a lysine residue. A second fibrinogen-binding peptide, which has the reverse sequence (YPRHG (SEQ ID NO: 19)), is attached at its amino terminal end by a linker to the lysine residue. The carboxy-terminal end of the second peptide comprises an amide group.

The linker allows the peptides to extend away from each other.

Peptide Conjugate No.1 or 2 (2 mg/ml) was mixed with Peptide Dendrimer No. 3 or 4, and fibrinogen, and the ability of the mixtures to copolymerise with fibrinogen was assessed using the same method described in Example 6, for a concentration of each dendrimer ranging from 0.025-0.5 mg/ml. FIG. 15 shows a plot of the clotting times (in seconds) obtained with increasing concentration of each different dendrimer.

The results show that, surprisingly, only mixtures containing Peptide Conjugate No.2 (i.e. with the B-knob peptides) and the dendrimer peptides were synergistic and increased activity, whereas mixtures containing the Peptide Conjugate No.1 (the A-kn [symbol] were not active when added to either Peptide Conjugate No.2 or the peptide dendrimers.

### EXAMPLE 11

### Ability of Peptide Dendrimers to Polymerise Fibrinogen in Human Plasma

The ability of several different peptide dendrimers (Nos. 4, 5, 8, 9, 10, 11, 12, 13) to polymerise fibrinogen in human plasma was tested.

30 μL of each dendrimer (at a concentration of 0.25 mg/ml) was added to 100 μL human plasma at 37° C., and polymerisation of fibrinogen was determined using a Sigma Amelung KC4 Delta coagulation analyzer.

The clotting times for each dendrimer are shown in FIG. 16, and show that peptide dendrimers Nos. 10, 11, 4, 12 and 13 were able to polymerise fibrinogen in human plasma, with dendrimer No. 12 being particularly effective (with a clotting time of less than one second). However, peptide dendrimers Nos. 5, 8, and 9 were not able to polymerise fibrinogen in human plasma.

### EXAMPLE 12

### Effect of Sterilisation on Ready-to-Use Peptide Dendrimer Formulations

This example describes the effect of Gamma irradiation on the haemostatic activity of peptide dendrimers formulated as a ready-to-use paste with hydrated gelatin.

2 ml of solution of Peptide Dendrimer No. 12 or 13 was mixed with SURGIFLO Haemostatic Matrix (a hydrated flowable gelatin matrix) to form a paste of each peptide. Each paste was sterilised by irradiation with ⁶⁰Co gamma rays at a dose of 30 kGy, and then stored at room temperature. Samples of the sterilised pastes were used for testing after storage for two and four weeks.

After storage, peptide dendrimers were extracted from each paste using 10 nM HEPES buffer. 30 μL of each extract (with a peptide concentration of about 0.25 mg/ml) was added to 100 μL of human fibrinogen at 3 mg/ml, and the ability of each dendrimer to polymerise fibrinogen (the ‘clotting’ activity) at 37° C. was determined using a Sigma Amelung KC4 Delta coagulation analyser. The polymerisation activity of non-irradiated control samples was also determined. The results are summarized in the Table below.

| | Clotting activity (seconds) | | |
|---|---|---|---|
| | | Storage for 2 | Storage for 4 |
| Peptide dendrimer | Non-irradiated | weeks post | weeks post |
| no. | control | irradiation | irradiation |
| 12 | 1 | 1 | 1 |
| 13 | 4.3 | 9.4 | 10 |

The results show that peptide dendrimers of the invention, formulated as a ready-to-use paste with hydrated gelatin, retain ability to polymerise fibrinogen after sterilization by irradiation.

1. A haemostatic device comprising a surgical fastener and a plurality of fibrinogen binding peptides immobilised to the fastener.

2. The device according to claim 1, wherein the plurality of fibrinogen-binding peptides are non-covalently immobilised to the fastener.

3. The device according to claim 1, wherein a plurality of carriers are immobilised to the fastener, and a plurality of fibrinogen-binding peptides are immobilised to each carrier.

4. The device according to claim 3, wherein the plurality of fibrinogen-binding peptides are covalently immobilised to each carrier.

5. The device according to claim 4, wherein each fibrinogen-binding peptide is covalently immobilised to the carrier by a non-peptide spacer.

6. The device according to claim 5, wherein the non-peptide spacer comprises a hydrophilic polymer.

7. The device according to claim 6, wherein the hydrophilic polymer comprises polyethylene glycol.

8. The device according to claim 3, wherein the carriers are soluble carriers.

9. The device according to claim 1, in which the fastener is manufactured from a resorbable material.

10. The device according to claim 9 in which the fastener comprises polyglactin, poliglecaprone, polydioxanone, animal gut or oxidised cellulose.

11. The device according to claim 1, wherein the fastener is manufactured from a non-resorbable material.

12. The device according to claim 11, wherein the fastener comprises polypropylene, polyester, nylon, silk or steel, preferably wherein the fastener comprises polypropylene.

13. The device according to claim 1, wherein the plurality of fibrinogen-binding peptides are covalently immobilised to the fastener.

14. The device according to claim 13, wherein the fastener comprises oxidised cellulose.

15. The device according to claim 1, wherein each fibrinogen binding peptide comprises the sequence Gly-(Pro/His)-Arg-Xaa (SEQ ID NO: 1) where Xaa is any amino acid and Pro/His means that either proline or histidine is present at that position.

16. The device according to claim 1 wherein each fibrinogen binding peptide comprises the sequence NH₂-Gly-(Pro/His)-Arg-Xaa (SEQ ID NO: 1) at its amino terminal end, where Xaa is any amino acid and Pro/His means that either proline or histidine is present at that position.

17. The device according to claim 1, wherein the fibrinogen-binding peptides are each 4-60 amino acid residues in length.

18. The device according to claim 1, wherein the fastener is a suture.

19. A kit for formation of a haemostatic device comprising a surgical fastener and, separately, a plurality of fibrinogen binding peptides for immobilising to the fastener.

20. The kit according to claim 19, which further comprises instructions to apply the fibrinogen binding peptides to the fastener to form the device, before application of the device to a patient.

21. A method of joining tissue which comprises applying a haemostatic device to a patient, the device as defined in claim 1.

22. A method of making a haemostatic device comprising immobilising a plurality of fibrinogen binding peptides to a surgical fastener.

23. The method according to claim 22, wherein the plurality of fibrinogen-binding peptides are immobilised to the fastener by non-covalently immobilising a haemostatic agent to the fastener, wherein the agent comprises a plurality of carriers and wherein there are a plurality of fibrinogen-binding peptides immobilised to each carrier.

24. The method according to claim 23, comprising contacting a solution of the agent with the fastener, and drying.

25. The method according to claim 23, comprising immobilising the agent to the fastener by thermal grafting.

26. The method according to claim 22, comprising covalently immobilising fibrinogen binding peptides to the fastener.

27.-28. (canceled)
